(19) United States
(12) Patent Application Publication
(10) Pub. No.: US 2008/0070910 A1
(43) Pub. Date: Mar. 20, 2008

(54) Therapeutic compounds and methods

(76) Inventors: Manoj C. Desai, Pleasant Hill, CA (US); Christopher P. Lee, San Francisco, CA (US); Hongtao Liu, Cupertino, CA (US); Sundaramoorthi Swaminathan, Burlingame, CA (US); Lianhong Xu, Palo Alto, CA (US)

Correspondence Address: GILEAD SCIENCES INC, 333 LAKESIDE DR, FOSTER CITY, CA, 94404, US

(21) Appl. No.: 11/880,743

(22) Filed: Jul. 24, 2007

Related U.S. Application Data
(60) Provisional application No. 60/832,882, filed on Jul. 24, 2006.

(51) Int. Cl. C07D 493/04; A61K 31/341; A61K 31/352; A61K 31/357; A61K 31/427; A61K 31/536; C12N 9/99; A61P 31/18; C07D 277/22; C07D 307/02; C07D 307/77; C07D 413/00

(52) U.S. Cl. 514/230.5; 435/184; 514/365; 514/466; 514/470; 514/471; 544/105; 548/204; 549/435; 549/464; 549/475

(57) ABSTRACT

The present invention provides compounds of Formula (I): 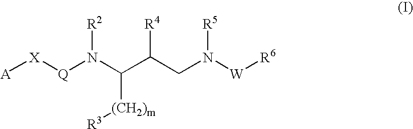
wherein A, X, Q, R²-R⁶, m, and W have the values described herein, as well as compositions comprising such compounds. The compounds are protease inhibitors and are useful for inhibiting the development of drug resistance in animals.

### CROSS-REFERENCE TO RELATED APPLICATIONS

This application claims priority to U.S. Provisional Application Ser. No. 60/832,882, filed Jul. 24, 2006. The content of this provisional application is herein incorporated by reference in its entirety for all purposes.

### FIELD OF THE INVENTION

The present invention relates to methods and compositions for inhibiting the development of drug resistance of HIV in an HIV-infected mammal.

### BACKGROUND OF THE INVENTION

Drug resistance is a common reason for drug failure. One of the most dramatic examples of drug failure due to resistance is in HIV therapy. Once HIV resistance is obtained to first-line therapy, the chances of future success are greatly diminished because of the development of multidrug cross resistance. Other diseases involving infectious agents (e.g., viruses, bacteria, protozoa, and prions) or other disease-causing cells (e.g., tumor cells) present similar challenges in that drug resistance is a primary cause of drug failure.

U.S. patent application Ser. No. 11/030,632, which was published on 21 Jul. 2005 as United States Patent Application Publication Number US 2005/0158713 relates to certain specific compounds of the following Formula (I): 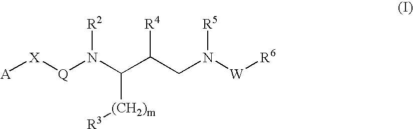
wherein R⁴ is OH, ═O, NH₂, or alkylamino; and wherein A, X, Q, R², R³, R⁵, R⁶, m, and W have the specific values described therein. The compounds are generally reported to be useful for preventing the development of drug resistance of HIV in an HIV infected mammal (see paragraph 0022). The synthesis of a few compounds of formula I wherein R⁴ is OH, was reported (for example, see Examples 11 and 12). Biological data was also reported for a few compounds of Formula (I) wherein R⁴ is OH (for example, see Examples 14, 15, 19, and 20). No compounds wherein R⁴ had a value other than OH were prepared; no biological data was reported for any compounds wherein R⁴ had a value other than OH.

### SUMMARY OF THE INVENTION

It has now been determined that compounds of Formula (I) wherein R⁴ is amino or substituted amino possess improved pharmacokinetic properties (e.g. liver stability) compared to compounds wherein R⁴ is OH. Accordingly, the invention provides a compound of the invention which is a compound of Formula (I): 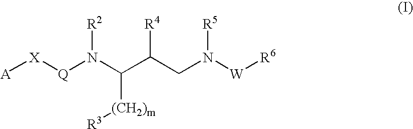
or a pharmaceutically acceptable salt thereof, wherein:

A is heteroaryl or a group having a formula:

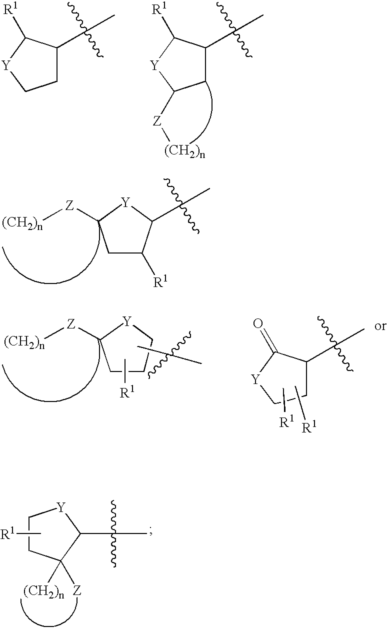

each R¹ is independently H, alkyl, alkenyl, alkynyl, cycloalkyl, cycloalkylalkyl, aryl, aralkyl, heterocycloalkyl, heterocycloalkylalkyl, heteroaryl, or heteroaralkyl, which R¹ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio;

Y and Z, the same or different, are independently selected from the group consisting of CH₂, O, S, SO, SO₂, NR⁸, R⁸C(O)N, R⁸C(S)N, R⁸OC(O)N, R⁸OC(S)N, R⁸SC(O)N, R⁸R⁹NC(O)N, and R⁸R⁹NC(S)N;

n is an integer from 1 to 5;

X is a covalent bond, CHR¹⁰, CHR¹⁰CH₂, CH₂CHR¹⁰, O, NR¹⁰, or S;

Q is C(O), C(S), or SO₂;

R² is H, alkyl, alkenyl, or alkynyl;

m is an integer from 0 to 6;

R³ is cycloalkyl, heterocycloalkyl, aryl, or heteroaryl, which R³ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, (R¹⁰O)₂P(═O)—, (R¹⁰O)₂P(═O)alkyl, (R¹⁰)₂P(═O)alkoxy, and heteroaralkylthio; wherein any cycloalkyl, heterocycloalkyl, aryl, and heteroaryl of the one or more substituents is optionally substituted with one or more halogen, cyano, nitro, carboxy, hydroxy, alkyl, haloalkyl, haloalkoxy, amino, alkylamino, alkanoylamino, alkoxy, alkylthio, or alkylamino;

R⁴ is ═NRₐ, or NRₐR_{b}; wherein Rₐ and R_{b} are each independently —V—R_{c}; wherein each V is independently a direct bond or —C(═O); and each R_{c} is independently H, hydroxy, alkyl, alkoxy, alkenyl, or alkynyl, and is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio;

R⁵ is H, C₁-C₆ alkyl, C₂-C₆ alkenyl, or (CH₂)_{q}R¹⁴, wherein q is an integer from 0 to 5; and R⁶ is cycloalkyl, heterocycloalkyl, aryl, or heteroaryl which R⁶ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio; or R⁵ and R⁶, together with the N—W bond of formula (I) comprise a macrocyclic ring which optionally comprises one or more additional heteroatoms in the macrocyclic ring;

R⁸ and R⁹ are each H, alkyl, alkenyl, or alkynyl;

each R¹⁰ is independently H, alkyl, alkenyl, or alkynyl;

R¹⁴ is cycloalkyl, heterocycloalkyl, aryl, or heteroaryl, which R¹⁴ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio; and

W is C(O), C(S), S(O), or SO₂.

The invention also provides a pharmaceutical composition comprising a compound of the invention and a pharmaceutically acceptable carrier.

The invention also provides a method for inhibiting the activity of a protease comprising contacting (in vitro or in vivo) the protease with an effective inhibitory amount of a compound of the invention.

The invention also provides a method for reducing the likelihood that a disease-causing entity (e.g. a virus) will develop drug resistance comprising, administering an effective amount of a compound of the invention to an animal that is infected with the disease causing entity.

The invention also provides the use of a compound of the invention to prepare a medicament useful for inhibiting the activity of a protease in an animal.

The invention also provides the use of a compound of the invention to prepare a medicament useful for reducing the likelihood that a disease-causing entity (e.g. a virus) will develop drug resistance (e.g. multi-drug resistance).

The invention also provides a method of administering a therapeutic compound that inhibits a biochemical target of a disease-causing replicating biological entity. The therapeutic compound, when administered in accordance with the method of the present invention, reduces the likelihood that the disease-causing entity will develop drug resistance. As such, the method of administering a therapeutic compound in accordance with the present invention improves the chances of long-term success in therapy.

The present invention also provides a method for inhibiting the development of drug resistance of HIV in an HIV-infected mammal comprising, administering a drug resistance-inhibiting effective amount of a compound of the invention.

### BRIEF DESCRIPTION OF THE FIGURES

FIG. 1 illustrates the synthesis of a particular sulfonamide core of a compound of the present invention.

FIG. 2 illustrates the synthesis of a bis-tetrahydrofuran ligand and the optical resolution thereof.

FIG. 3A illustrates the synthesis of a compound of the present invention via coupling of a bis-tetrahydrofuran and a sulfonamide.

FIG. 3B illustrates the synthesis of a compound of the present invention via coupling of a bis-tetrahydrofuran and a sulfonamide.

### DETAILED DESCRIPTION OF THE INVENTION

As utilized herein, the term “alkyl” means a straight-chain or branched alkyl radical containing from about 1 to about 20 carbon atoms chain, preferably from about 1 to about 10 carbon atoms, more preferably from about 1 to about 8-carbon atoms, still more preferably from about 1 to about 6 carbon atoms. Examples of such substituents include methyl, ethyl, propyl, isopropyl, n-butyl, sec-butyl, isobutyl, tert-butyl, pentyl, isoamyl, hexyl, octyl, dodecanyl, and the like.

The term “alkenyl” means a straight-chain or branched-chain alkenyl radical having one or more double bonds and containing from about 2 to about 20 carbon atoms chain, preferably from about 2 to about 10 carbon atoms, more preferably from about 2 to about 8 carbon atoms, still more preferably from about 2 to about 6 carbon atoms. Examples of such substituents include vinyl, allyl, 1,4-butadienyl, isopropenyl, and the like.

The term “alkynyl” means a straight-chain or branched-chain alkynyl radical having one or more triple bonds and containing from about 2 to about 20 carbon atoms chain, preferably from about 2 to about 10 carbon atoms, more preferably from about 2 to about 8 carbon atoms, still more preferably from about 2 to about 6 carbon atoms. Examples of such radicals include ethynyl, propynyl (propargyl), butynyl, and the like.

The term “alkanoyl” means a group alkyl-C(═O)—. Examples include acyl, propanoyl, isopropanoyl, and butanoyl.

The term “amino” means NH₂.

The term “alkoxy” means an alkyl ether radical, wherein the term “alkyl” is defined as above. Examples of alkoxy radicals include methoxy, ethoxy, n-propoxy, isopropoxy, n-butoxy, isobutoxy, sec-butoxy, tert-butoxy, hexyloxy, and the like.

The term “alkoxycarbonyl” means a group alkoxy-C(═O)—. Examples include tert-butoxycarbonyl, methoxycarbonyl, and ethoxycarbonyl.

The term “alkylthio” means an alkyl thioether radical, wherein the term “alkyl” is defined as above. Examples of alkylthio radicals include methylthio (SCH₃), ethylthio (SCH₂CH₃), n-propylthio, isopropylthio, n-butylthio, isobutylthio, sec-butylthio, tert-butylthio, n-hexylthio, and the like.

The term “alkylamino” means an alkyl amine radical, wherein the term “alkyl” is defined as above. Examples of alkylamino radicals include methylamino (NHCH₃), ethylamino (NHCH₂CH₃), n-propylamino, isopropylamino, n-butylamino, isobutylamino, sec-butylamino, tert-butylamino, n-hexylamino, and the like.

The term “alkanoylamino” means an amino group where one nitrogen has been replaced with an alkanoyl group. Examples include acylamino, propanoylamino, and isopropanoylamino.

The term “alkoxycarbonylamino” means an amino group where one hydrogen has been replaced with an alkoxycarbonyl group. Examples include tert-butoxycarbonylamino and methoxycarbonylamino.

The term “cycloalkyl” means a monocyclic or a polycyclic alkyl radical defined by one or more alkyl carbocyclic rings, which can be the same or different when the cycloalkyl is a polycyclic radical having 3 to about 10 carbon atoms in the carbocyclic skeleton in each ring, preferably about 4 to about 7 carbon atoms, more preferably 5 to 6 carbons atoms. Examples of monocyclic cycloalkyl radicals include cyclopropyl, cyclobutyl, cyclopentyl, cyclohexyl, cycloheptyl, cyclodecyl, and the like. Examples of polycyclic cycloalkyl radicals include decahydronaphthyl, bicyclo[5.4.0]undecyl, adamantyl, and the like.

The term “cycloalkylalkyl” means an alkyl radical as defined herein, where one or more hydrogen atom on the alkyl radical is replaced by a cycloalkyl radical as defined herein. Examples of cycloalkylalkyl radicals include cyclohexylmethyl, 3-cyclopentylbutyl, and the like.

The term “haloalkyl” means an alkyl group where one or more hydrogens have been replaced with independently selected halo atoms. Examples include fluoromethyl, difluoromethyl, trifluoromethyl, 2-fluoroethyl, 2,2-difluoroethyl, 2,2,2-trifluoroethyl, 2-chloro-2-fluoroethyl, and perfluoroethyl.

The term “haloalkoxy” means an alkoxy group where one or more hydrogens have been replaced with independently selected halo atoms. Examples include fluoromethoxy, difluoromethoxy, trifluoromethoxy, 2-fluoroethoxy, 2,2-difluoroethoxy, 2,2,2-trifluoroethoxy, 2-chloro-2-fluoroethoxy, and perfluoroethoxy.

The term “heterocycloalkyl” means a cycloalkyl radical as defined herein (including polycyclics), wherein at least one carbon which defines the carbocyclic skeleton is replaced with a heteroatom such as, for example, O, N, or S, optionally comprising one or more double-bond within the ring, provided the ring is not heteroaryl as defined herein. The heterocycloalkyl preferably has 3 to about 10 atoms (members) in the carbocyclic skeleton of each ring, preferably about 4 to about 7 atoms, more preferably 5 to 6 atoms. Examples of heterocycloalkyl radicals include epoxy, aziridyl, oxetanyl, tetrahydrofuranyl, dihydrofuranyl, piperadyl, piperidinyl, pyperazyl, piperazinyl, pyranyl, morpholinyl, and the like.

The term “heterocycloalkylalkyl” means an alkyl radical as defined herein, in which at least one hydrogen atom on the alkyl radical is replace by a heterocycloalkyl radical as defined herein. Examples of heterocycloalkylalkyl radicals include 2-morpholinomethyl, 3-(4-morpholino)-propyl, 4-(2-tetrahydrofuranyl)-butyl, and the like.

The term “aryl” refers to an aromatic carbocyclic radical, as commonly understood in the art, and includes monocyclic and polycyclic aromatics such as, for example, phenyl and naphthyl radicals. Each aryl may optionally be “substituted” as described herein, and may also be substituted with methylenedioxy, ethylenedioxy, —N(R)CH₂CH₂O—, or —N(R)C(═O)CH₂O—, wherein R is H or alkyl.

The term “aryloxy” means a group aryl-O—. Examples of aryloxy radicals include phenoxy, naphthyloxy, 4-fluorophenoxy, and the like.

The term “arylamino” means a group aryl-NH—. Examples of arylamino radicals include phenylamino, naphthylamino, 3-nitrophenylamino, 4-aminophenylamino, and the like.

The term “arylthio” means a group aryl-S—. Examples of arylthio radicals include phenylthio, naphthylthio, 3-nitrophenylthio, 4-thiophenylthio, and the like.

The term “aralkyl” means a group aryl-alkyl. Examples of aralkyl radicals include benzyl, phenethyl, 3-(2-naphthyl)-butyl, and the like.

The term “aryloxyalkyl” means a group aryloxy-alkyl. Examples of aryloxyalkyl radicals include phenoxyethyl, 4-(3-aminophenoxy)-1-butyl-, and the like.

The term “arylaminoalkyl” means a group arylamino-alkyl. Examples of arylaminoalkyl radicals include phenylaminoethyl, 4-(3-methoxyphenylamino)-1-butyl, and the like.

The term “aralkoxy” means a group aryl-alkoxy. Examples of aralkoxy radicals include benzyloxy, 2-phenylethoxy, 2-phenyl-1-propoxy, and the like.

The term “(aryloxy)alkoxy” means a group aryloxy-alkoxy. Examples of (aryloxy)alkoxy radicals include 2-phenoxyethoxy, 4-(3-aminophenoxy)-1-butoxy, and the like.

The term “(arylamino)alkoxy” means a group arylamino-alkoxy. Examples of (arylamino)alkoxy radicals include 2-(phenylamino)-ethoxy, 2-(2-naphthyl-amino)-1-butoxy, and the like.

The term “(arylthio)alkoxy” means a group arylthio-alkoxy. Examples of (arylthio)alkoxy radicals include 2-(phenylthio)-ethoxy, and the like.

The term “aralkylamino” means a group aryl-alkylamino. Examples of aralkylamino radicals include 2-phenethylamino, 4-phenyl-n-butylamino, and the like.

The term “(aryloxy)alkylamino” means a group aryloxy-alkylamino. Examples of (aryloxy)alkylamino radicals include 3-phenoxy-n-propylamino, 4-phenoxybutylamino, and the like.

The term “(arylamino)alkylamino” means a group arylamino-alkylamino. Examples of (arylamino)alkylamino radicals include 3-(naphthylamino)-1-propylamino, 4-(phenylamino)-1-butylamino, and the like.

The term “(arylthio)alkylamino” means a group arylthio-alkylamino. Examples of (arylthio)alkylamino radicals include 2-(phenylthio)-ethylamino, and the like.

The term “aralkylthio” means a group aryl-alkylthio. Examples of aralkylthio radicals include 3-phenyl-2-propylthio, 2-(2-naphthyl)-ethylthio, and the like.

The term “(aryloxy)alkylthio” means a group aryloxy-alkylthio. Examples of (aryloxy)alkylthio radicals include 3-phenoxypropylthio, 4-(2-fluorophenoxy)-butylthio, and the like.

The term “(arylamino)alkylthio” means a group arylamino-alkylthio. Examples of (arylamino)alkylthio radicals include 2-(phenylamino)-ethylthio, 3-(2-naphthylamino)-n-propylthio, and the like.

The term “(arylthio)alkylthio” means a group arylthio-alkylthio. Examples of (arylthio)alkylthio radicals include 2-(naphthylthio)-ethylthio, 3-(phenylthio)-propylthio, and the like.

The term “heteroaryl” means an optionally substituted radical defined by an aromatic heterocyclic ring as commonly understood in the art, including monocyclic radicals such as, for example, imidazole, thiazole, pyrazole, pyrrole, furane, pyrazoline, thiophene, oxazole, isoxazol, pyridine, pyridone, pyrimidine, pyrazine, and triazine radicals, and also including polycyclics such as, for example, quinoline, isoquinoline, indole, and benzothiazole radicals.

The term “heteroaryloxy” means a group heteroaryl-O—. Heteroaryloxy radicals include, for example, 4-pyridyloxy, 5-quinolyloxy, and the like.

The term “heteroarylamino” means a group heteroaryl-NH— as defined herein, wherein a hydrogen atom on the heteroaryl ring is replaced by an nitrogen. Heteroarylamino radicals include, for example, 4-thiazolylamino, 2-pyridylamino, and the like.

The term “heteroarylthio” means a group heteroaryl-S— as defined herein, wherein a hydrogen atom on the heteroaryl ring is replaced by a sulfur. Heteroarylthio radicals include, for example, 3-pyridylthio, 3-quinolylthio, 4-imidazolylthio, and the like.

The term “heteroaralkyl” means a group heteroaryl-alkyl. Examples of heteroaralkyl radicals include 2-pyridylmethyl, 3-(4-thiazolyl)-propyl, and the like.

The term “heteroaralkoxy” means a group heteroaryl-alkoxy. Examples of heteroaralkoxy radicals include 2-pyridylmethoxy, 4-(1-imidazolyl)-butoxy, and the like.

The term “heteroaralkylamino” means a group heteroaryl-alkylamino. Examples of heteroaralkylamino radicals include 4-pyridylmethylamino, 3-(2-furanyl)-propylamino, and the like.

The term “heteroaralkylthio” means a group heteroaryl-alkylthio. Examples of heteroaralkylthio radicals include 3-pyridylmethylthio, 3-(4-thiazolyl)-propylthio, and the like.

In one embodiment of the invention, when a group is “substituted,” one or more hydrogens on the group have been replaced with substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylamino, arylthio, aralkyl, aryloxyalkyl, arylaminoalkyl, aralkoxy, (aryloxy)alkoxy, (arylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio.

Specific and preferred values listed below for radicals, substituents, and ranges, are for illustration only; they do not exclude other defined values or other values within defined ranges for the radicals and substituents

In one embodiment of the invention the compound of formula I is selected from the following compounds and pharmaceutically acceptable salts thereof: 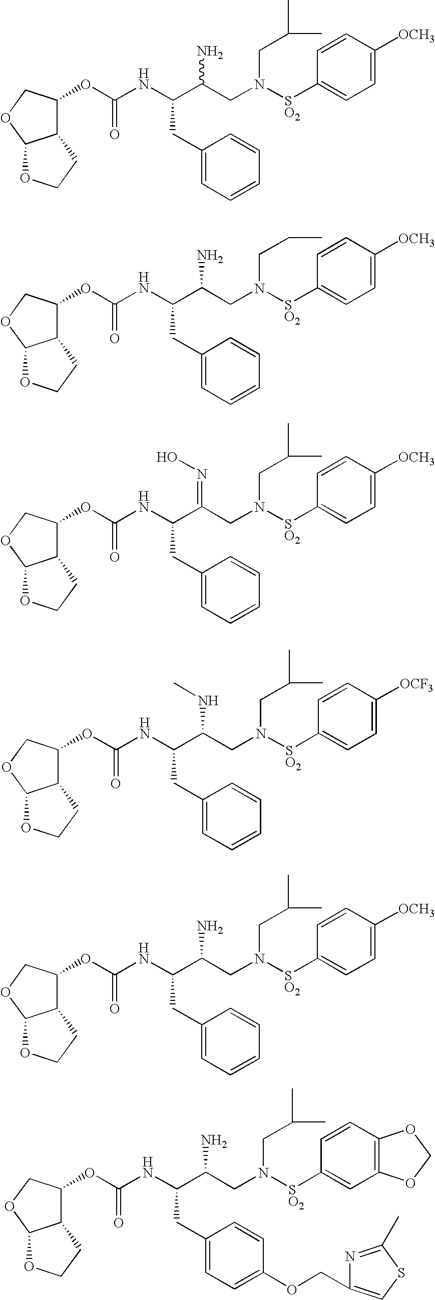 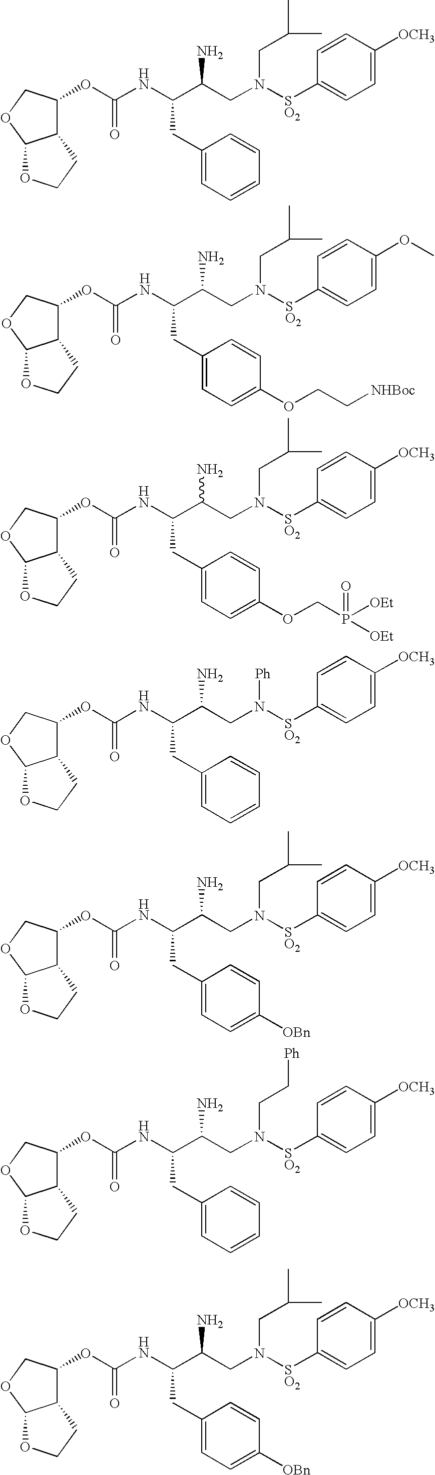 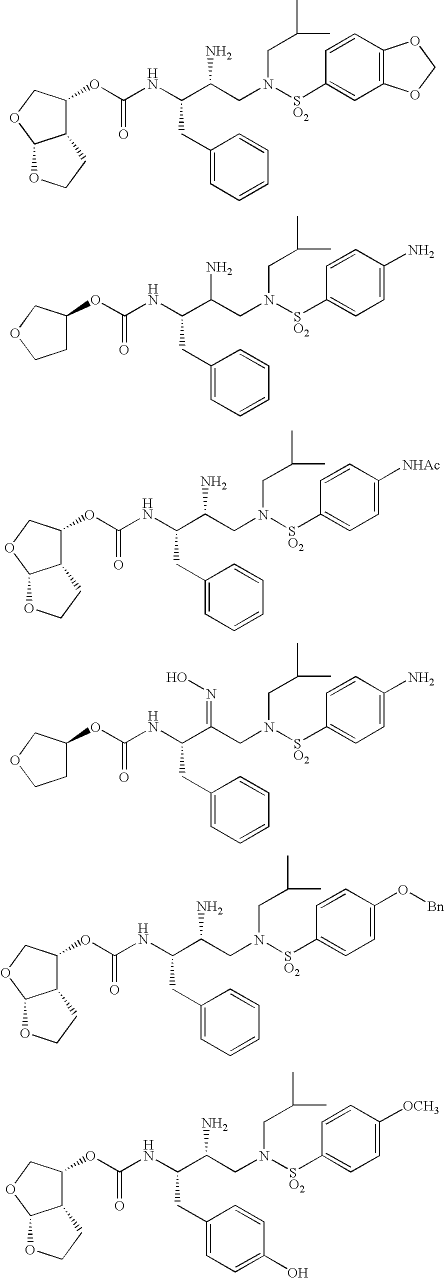 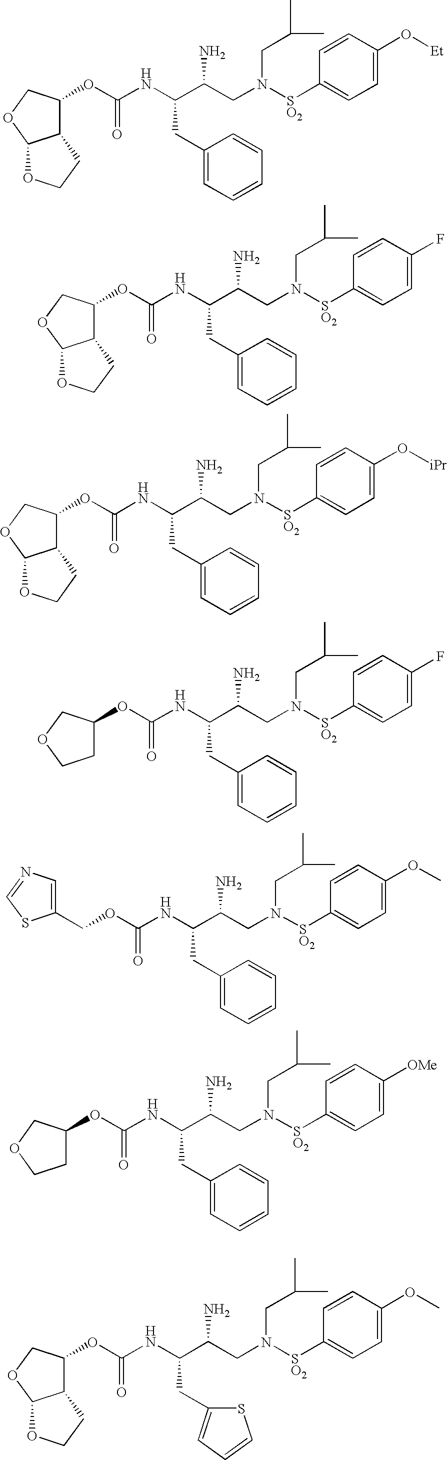 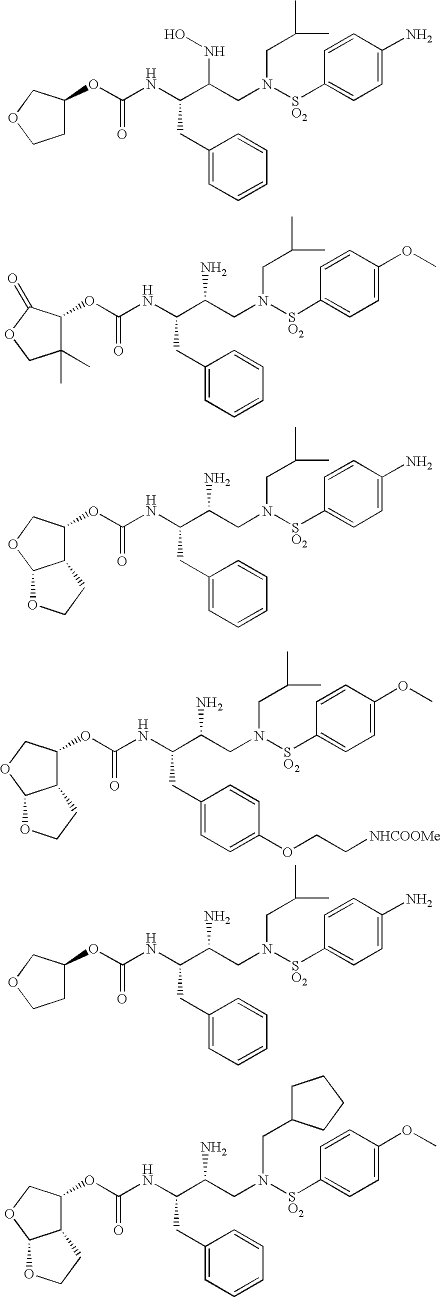 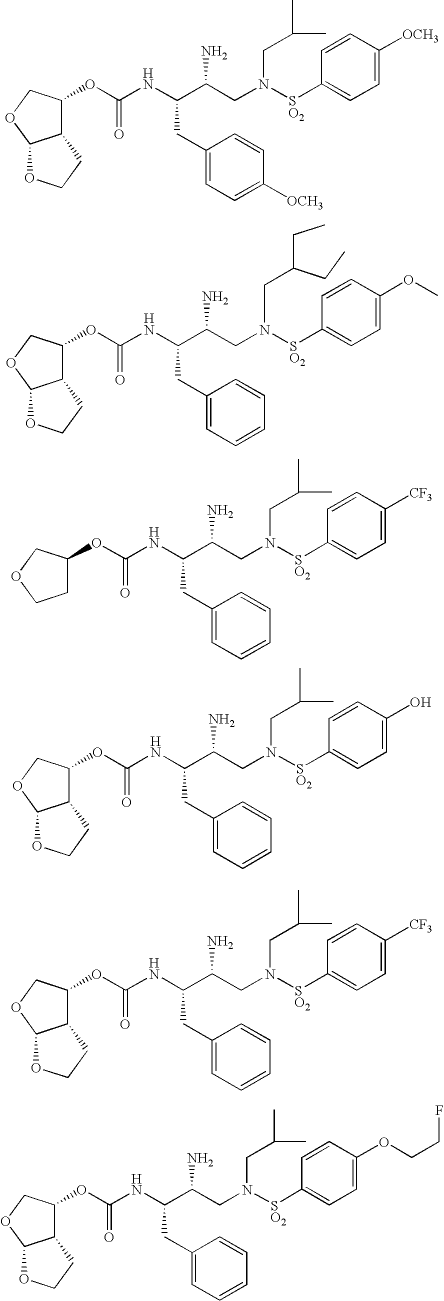 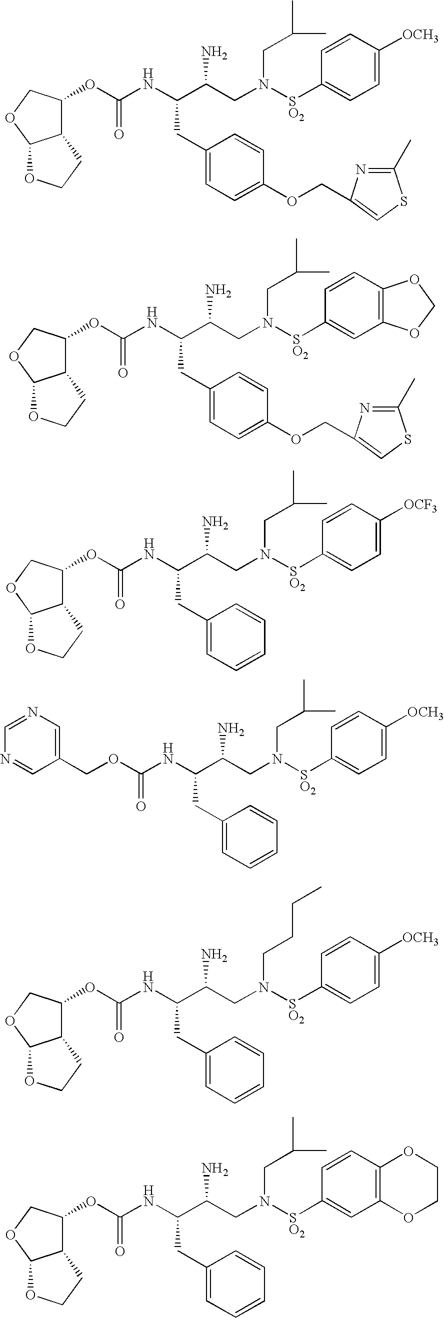 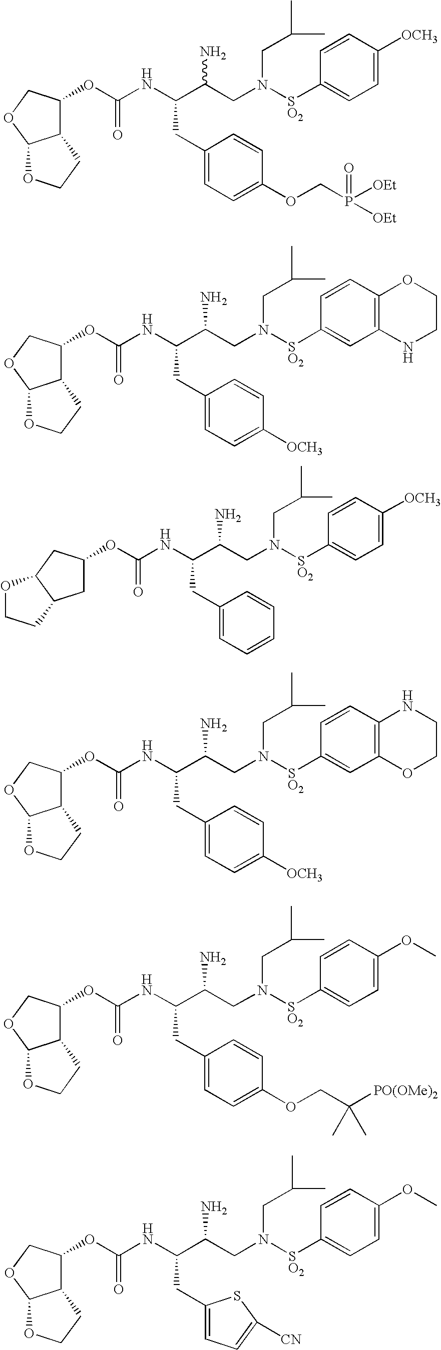 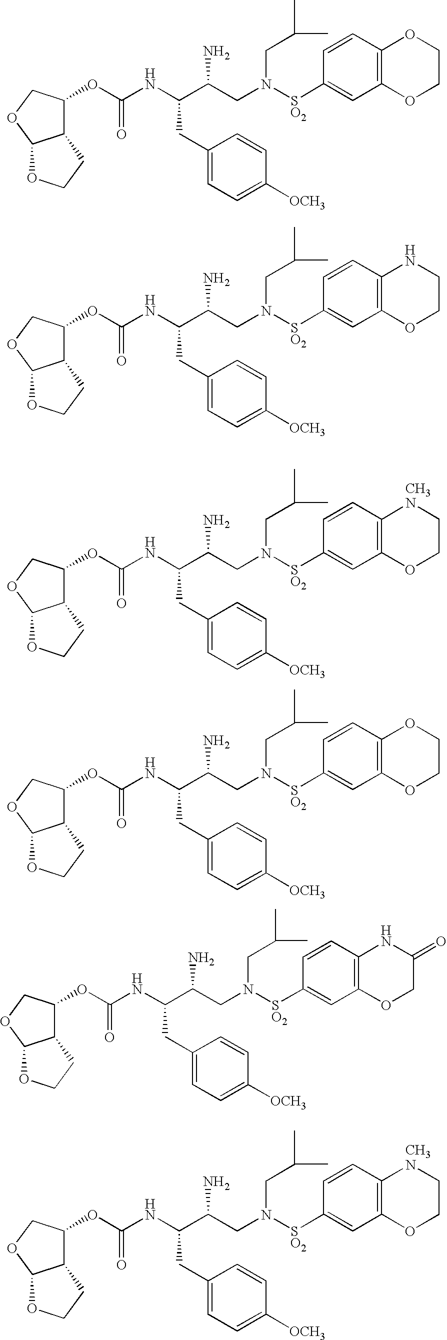 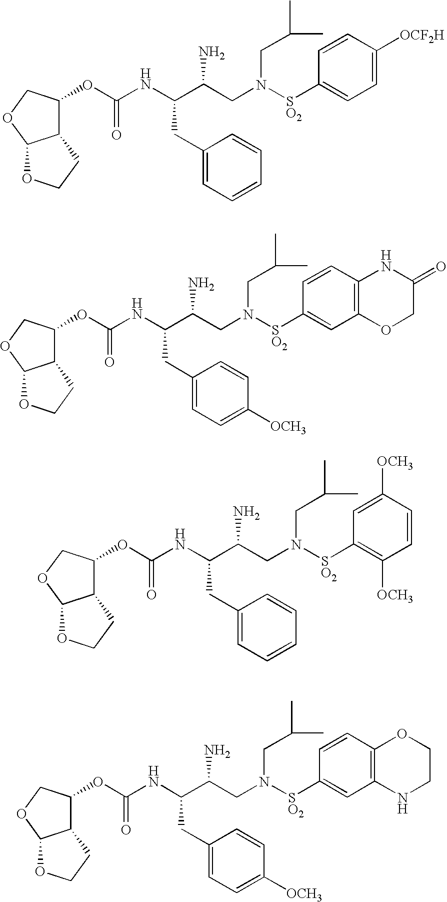
Methods of the Invention

The present invention provides a method of administering a therapeutic compound, which method increases the chances of successful long-term therapy. In one embodiment, the present invention provides a method of administering a therapeutic compound that inhibits a biochemical target of a replicating disease-causing replicating biological entity (disease causing predecessor).

In one embodiment, the disease-causing replicating biological entity is an infectious microorganism, for example, a virus, a fungus, a protozoa, or a bacterium. When the infectious microorganism is a virus, it can be a retrovirus, such as HIV-1 or HIV-2. When the infectious microorganism is a protozoa, it can be a malarial parasite, such as a plasmodium species.

In another embodiment, the disease-causing replicating biological entity can be a cancer cell, which can be a rapidly growing tumor cell, for example, a rapidly growing cancer cell found in breast cancer, colon cancer, lung cancer, or the like.

The present invention also provides a method of preventing the emergence of drug resistance in an HIV-infected mammal that includes administering a drug resistance-inhibiting effective amount of a compound represented by the Formula (I): 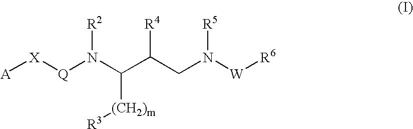
or a pharmaceutically acceptable salt, prodrug, or ester thereof, wherein:

A is a group having a formula:

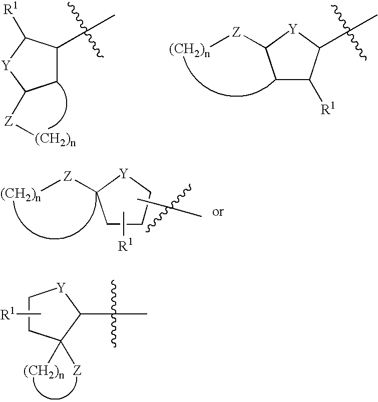

R¹ is H, alkyl, alkenyl, alkynyl, cycloalkyl, cycloalkylalkyl, aryl, aralkyl, heterocycloalkyl, heterocycloalkylalkyl, heteroaryl, or heteroaralkyl, wherein one or more hydrogen atoms can optionally be replaced with a substituent selected from the group consisting of OR⁷, SR⁷, CN, NO₂, N₃, and a halogen;

Y and Z, the same or different, are independently selected from the group consisting of CH₂, O, S, SO, SO₂, NR⁸, R⁸C(O)N, R⁸C(S)N, R⁸OC(O)N, R⁸OC(S)N, R⁸SC(O)N, R⁸R⁹NC(O)N, and R⁸R⁹NC(S)N;

n is an integer from 1 to 5;

X is a covalent bond, CHR¹⁰, CHR¹⁰CH₂, CH₂CHR¹⁰, O, NR¹⁰, or S;

Q is C(O), C(S), or SO₂;

R² is H, alkyl, alkenyl, or alkynyl;

m is an integer from 0 to 6;

R³ is cycloalkyl, heterocycloalkyl, aryl, or heteroaryl wherein one or more hydrogen atoms can optionally be replaced with a substituent independently selected from the group consisting of alkyl, (CH₂)ₚR¹¹, OR¹², SR¹², CN, N₃, NO₂, NR¹²R¹³, C(O)R¹², C(S)R¹², CO₂R¹², C(O)SR¹², C(O)NR¹²R¹³, C(S)NR¹²R¹³, NR¹²C(O)R¹³, NR¹²C(S)R¹³, NR¹²CO₂R¹³, NR¹²C(O)SR¹³, and halogen;

R⁴ is NRₐR_{b}; wherein Rₐ and R_{b} are each independently —V—R_{c}; wherein each V is independently a direct bond or —C(═O); and each R_{c} is independently H, alkyl, alkoxy, alkenyl, or alkynyl, which alkyl, alkoxy, alkenyl, or alkynyl is optionally substituted;

R⁵ is H, C₁-C₆ alkyl, C₂-C₆ alkenyl, or (CH₂)_{q}R¹⁴, wherein q is an integer from 0 to 5, and R⁶ is cycloalkyl, heterocycloalkyl, aryl, or heteroaryl wherein one or more hydrogen atoms can optionally be replaced with a substituent independently selected from the group consisting of halogen, OR¹⁵, SR¹⁵, S(O)R¹⁵, SO₂R¹⁵, SO₂NR¹⁵R¹⁶, SO₂N(OH)R¹⁵, CN, CR¹⁵═NR¹⁶, CR¹⁵═N(OR¹⁶), NO₂, NR¹⁵R¹⁶, N(OH)R¹⁵, C(O)R¹⁵, C(S)R¹⁵, CO₂R¹⁵, C(O)SR¹⁵, C(O)NR¹⁵R¹⁶, C(S)NR¹⁵R¹⁶, C(O)N(OH)R¹⁵, C(S)N(OH)R¹⁵, NR¹⁵C(O)R¹⁶, NR¹⁵C(S)R¹⁶, N(OH)C(O)R¹⁵, N(OH)C(S)R¹⁵, NR¹⁵CO₂R¹⁶, N(OH)CO₂R¹⁵, NR¹⁵C(O)SR¹⁶, NR¹⁵C(O)NR¹⁶R¹⁷, NR¹⁵C(S)NR¹⁶R¹⁷, N(OH)C(O)NR¹⁵R¹⁶, N(OH)C(S)NR¹⁵R¹⁶, NR¹⁵C(O)N(OH)R¹⁶, NR¹⁵C(S)N(OH)R¹⁶, NR¹⁵SO₂R¹⁶, NHSO₂NR¹⁵R¹⁶, NR¹⁵SO₂NHR¹⁶, P(O)(OR¹⁵)(OR¹⁶), alkyl, alkoxy, alkylthio, alkylamino, cycloalkyl, cycloalkylalkyl, heterocycloalkyl, heterocycloalkylalkyl, aryl, aryloxy, arylamino, arylthio, aralkyl, aryloxyalkyl, arylaminoalkyl, aralkoxy, (aryloxy)alkoxy, (arylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio; or R⁵ and R⁶ are covalently bonded such that R⁵ and R⁶ together with the N—W bond of formula (I) comprise a 12-18 membered ring that can comprise at least one additional heteroatom (e.g. N, O, or S) in the ring other than the nitrogen of the N—W bond;

W is C(O), C(S), S(O), or SO₂;

R⁷ is H, alkyl, alkenyl, or alkynyl;

R⁸ and R⁹ are independently selected from the group consisting of H, alkyl, alkenyl, and alkynyl;

R¹⁰ is H, alkyl, alkenyl, or alkynyl;

p is an integer from 0 to 5;

R¹¹ is cycloalkyl, heterocycloalkyl, aryl, or heteroaryl wherein one or more hydrogen atoms can optionally be replaced with a substituent independently selected from the group consisting of a halogen, OH, OCH₃, NH₂, NO₂, SH, and CN;

R¹² and R¹³ are independently selected from the group consisting of H, alkyl, alkenyl, and alkynyl; and

R¹⁴ is cycloalkyl, heterocycloalkyl, aryl, or heteroaryl wherein one or more hydrogen atoms can optionally be replaced with a substituent selected from the group consisting of a halogen, OH, OCH₃, NH₂, NO₂, SH, and CN;

R¹⁵, R¹⁶, and R¹⁷ are H, unsubstituted alkyl, or unsubstituted alkenyl.

A specific value for R³ is cycloalkyl, heterocycloalkyl, aryl, or heteroaryl wherein at least one hydrogen atom is optionally replaced with a substituent independently selected from the group consisting of alkyl, (CH₂)ₚR¹¹, OR¹², SR¹², CN, N₃, NO₂, NR¹²R¹³, C(O)R¹², C(S)R¹², CO₂R¹², C(O)SR¹², C(O)NR¹²R¹³, C(S)NR¹²R¹³, NR¹²C(O)R¹³, NR¹²C(S)R¹³, NR¹²CO₂R¹³, NR¹²C(O)SR¹³, and halogen.

A specific value for R⁵ is H, C₁-C₆ alkyl, C₂-C₆ alkenyl, or (CH₂)_{q}R¹⁴, wherein q is an integer from 0 to 5, R¹⁴ is cycloalkyl, heterocycloalkyl, aryl, or heteroaryl wherein at least one hydrogen atom is optionally replaced with a substituent selected from the group consisting of a halogen, OH, OCH₃, NH₂, NO₂, SH, and CN.

A specific value for R⁶ is cycloalkyl, heterocycloalkyl, aryl, or heteroaryl wherein at least one hydrogen atom is optionally replaced with a substituent independently selected from the group consisting of halogen, OR¹⁵, SR¹⁵, S(O)R¹⁵, SO₂R¹⁵, SO₂NR¹⁵R¹⁶, SO₂N(OH)R¹⁵, CN, CR¹⁵═NR¹⁶, CR¹⁵═N(OR¹⁶), N₃, NO₂, NR¹⁵R¹⁶, N(OH)R¹⁵, C(O)R¹⁵, C(S)R¹⁵, CO₂R¹⁵, C(O)SR¹⁵, C(O)NR¹⁵R¹⁶, C(S)NR¹⁵R¹⁶, C(O)N(OH)R¹⁵, C(S)N(OH)R¹⁵, NR¹⁵C(O)R¹⁵, NR¹⁵C(S)R¹⁶, N(OH)C(O)R¹⁵, N(OH)C(S)R¹⁵, NR¹⁵CO₂R¹⁶N(OH)CO₂R¹⁵, NR¹⁵C(O)SR¹⁶, NR¹⁵C(O)NR¹⁶R¹⁷, NR¹⁵C(S)NR¹⁶R¹⁷, N(OH)C(O)NR¹⁵R¹⁶, N(OH)C(S)NR¹⁵R¹⁶, NR¹⁵C(O)N(OH)R¹⁶, NR¹⁵C(S)N(OH)R¹⁶, NR¹⁵SO₂R¹⁶, NHSO₂NR¹⁵R¹⁶, NR¹⁵SO₂NHR¹⁶, P(O)(OR¹⁵)(OR¹⁶), alkyl, alkoxy, alkylthio, alkylamino, cycloalkyl, cycloalkylalkyl, heterocycloalkyl, heterocycloalkylalkyl, aryl, aryloxy, arylamino, arylthio, aralkyl, aryloxyalkyl, arylaminoalkyl, aralkoxy, (aryloxy)alkoxy, (arylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio.

A specific value for R¹¹ is cycloalkyl, heterocycloalkyl, aryl, or heteroaryl wherein at least one hydrogen atom is optionally replaced with a substituent independently selected from the group consisting of a halogen, OH, OCH₃, NH₂, NO₂, SH, and CN.

A specific group of compounds are compounds wherein when at least one hydrogen atom of R⁶ is optionally replaced with a substituent other than halogen, OR¹⁵, SR¹⁵, S(O)R¹⁵, SO₂R¹⁵, SO₂NR¹⁵R¹⁶, SO₂N(OH)R¹⁵, CN, CR¹⁵═NR¹⁶, CR¹⁵═N(OR¹⁶), N₃, NO₂, NR¹⁵R¹⁶, N(OH)R¹⁵, C(O)R¹⁵, C(S)R¹⁵, CO₂R¹⁵, C(O)SR¹⁵, C(O)NR¹⁵R¹⁶, C(S)NR¹⁵R¹⁶, C(O)N(OH)R¹⁵, C(S)N(OH)R¹⁵, NR¹⁵C(O)R¹⁶, NR¹⁵C(S)R¹⁶, N(OH)C(O)R¹⁵, N(OH)C(S)R¹⁵, NR¹⁵CO₂R¹⁶, N(OH)CO₂R¹⁵, NR¹⁵C(O)SR¹⁶, NR¹⁵C(O)NR¹⁶R¹⁷, NR¹⁵C(S)NR¹⁶R¹⁷, N(OH)C(O)NR¹⁵R¹⁶, N(OH)C(S)NR¹⁵R¹⁶, NR¹⁵C(O)N(OH)R¹⁶, NR¹⁵C(S)N(OH)R¹⁶, NR¹⁵SO₂R¹⁶, NHSO₂NR¹⁵R¹⁶, NR¹⁵SO₂NHR¹⁶, or P(O)(OR¹⁵)(OR¹⁶), then at least one hydrogen atom on said substituent is optionally replaced with halogen, OR¹⁵, SR¹⁵, S(O)R¹⁵, SO₂R¹⁵, SO₂NR¹⁵R¹⁶, SO₂N(OH)R¹⁵, CN, CR¹⁵═NR¹⁶, CR¹⁵═N(OR¹⁶), N₃, NO₂, NR¹⁵R¹⁶, N(OH)R¹⁵, C(O)R¹⁵, C(S)R¹⁵, CO₂R¹⁵, C(O)SR¹⁵, C(O)NR¹⁵R¹⁶, C(S)NR¹⁵R¹⁶, C(O)N(OH)R¹⁵, C(S)N(OH)R¹⁵, NR¹⁵C(O)R¹⁶, NR¹⁵C(S)R¹⁶, N(OH)C(O)R¹⁵, N(OH)C(S)R¹⁵, NR¹⁵CO₂R¹⁶, N(OH)CO₂R¹⁵, NR¹⁵C(O)SR¹⁶, NR¹⁵C(O)NR¹⁶R¹⁷, NR¹⁵C(S)NR¹⁶R¹⁷, N(OH)C(O)NR¹⁵R¹⁶, N(OH)C(S)NR¹⁵R¹⁶, NR¹⁵C(O)N(OH)R¹⁶, NR¹⁵C(S)N(OH)R¹⁶, NR¹⁵SO₂R¹⁶, NHSO₂NR¹⁵R¹⁶, NR¹⁵SO₂NHR¹⁶, or P(O)(OR¹⁵)(OR⁶).

In one specific embodiment of the invention, A is a group of the formula: 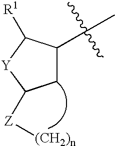
R¹ is H alkyl, alkenyl, cycloalkyl, cycloalkylalkyl, aryl, aralkyl, heterocycloalkyl, heterocycloalkylalkyl, heteroaryl, or a heteroaralkyl, wherein one or more hydrogen atoms can optionally be replaced with a substituent independently selected from the group consisting of OR⁷, SR⁷, CN, NO₂, N₃, and a halogen, wherein R⁷ is H, an unsubstituted alkyl, or an unsubstituted alkenyl; Y and Z are the same or different and are independently selected from the group consisting of CH₂, O, S, SO, SO₂, NR⁸, R⁸C(O)N, R⁸C(S)N, R⁸OC(O)N, R⁸SC(S)N, R⁸SC(O)N, R⁸R⁹NC(O)N, and R⁸R⁹NC(S)N, wherein R⁸ and R⁹ are independently selected from the group consisting of H, an unsubstituted alkyl, and an unsubstituted alkenyl; n is an integer from 1 to 5; X is a covalent bond, CHR¹⁰, CHR¹⁰CH₂, CH₂CHR¹⁰, O, NR¹⁰, or S, wherein R¹⁰ is H, an unsubstituted alkyl, or an unsubstituted alkenyl; R² is H, a C₁-C₆ alkyl radical, or a C₂-C₆ alkenyl radical; R¹² and R¹³, as defined with respect to R³, are independently selected from the group consisting of H, an unsubstituted alkyl, and an unsubstituted alkenyl radical; R⁴ is H, NH₂, or NHCH₃; W is C(O), C(S), or SO₂; and R⁶ is a cycloalkyl, heterocycloalkyl, aryl, or heteroaryl radical wherein one or more hydrogen atoms can optionally be replaced with a substituent independently selected from the group consisting of a halogen, OR¹⁵, SR¹⁵, CN, N₃, NO₂, NR¹⁵R¹⁶, C(O)R¹⁵, C(S)R¹⁵, CO₂R¹⁵, C(O)SR¹⁵, C(O)NR¹⁵R¹⁶, C(S)NR¹⁵R¹⁶, NR¹⁵C(O)R¹⁶, NR¹⁵C(S)R¹⁶, NR¹⁵CO₂R¹⁶, NR⁵C(O)SR¹⁶, NR¹⁵C(O)NR¹⁶R¹⁷, and NR¹⁵C(S)NR¹⁶R¹⁷, an alkyl, an alkoxy, an alkylthio, an alkylamino, a cycloalkyl, a cycloalkylalkyl, a heterocycloalkyl, a heterocycloalkylalkyl, an aryl, an aryloxy, an arylamino, an arylthio, an aralkyl, an aryloxyalkyl, an arylaminoalkyl, an aralkoxy, an (aryloxy)alkoxy, an (arylamino)alkoxy, an (arylthio)alkoxy, an aralkylamino, an (aryloxy)alkylamino, an (arylamino)alkylamino, an (arylthio)alkylamino, an aralkylthio, an (aryloxy)alkylthio, an (arylamino)alkylthio, an (arylthio)alkylthio, a heteroaryl, a heteroaryloxy, a heteroarylamino, a heteroarylthio, a heteroaralkyl, a heteroaralkoxy, a heteroaralkylamino, and a heteroaralkylthio, wherein R¹⁵, R¹⁶ and R¹⁷ are H, an unsubstituted alkyl, and an unsubstituted alkenyl, such that when at least one hydrogen atom of R⁶ is optionally substituted with a substituent other than a halogen, OR¹⁵, SR¹⁵, CN, N₃, NO₂, NR⁵R⁶, C(O)R¹⁵, C(S)R¹⁵, CO₂R¹⁵, C(O)SR¹⁵, C(O)NR¹⁵R¹⁶, C(S)NR¹⁵R¹⁶, NR¹⁵C(O)R¹⁶, NR¹⁵C(S)R¹⁶, NR¹⁵CO₂R¹⁶, NR¹⁵C(O)SR¹⁶, NR¹⁵C(O)NR¹⁶R¹⁷, or NR¹⁵C(S)NR¹⁶R¹⁷, at least one hydrogen atom on said substituent attached to R⁶ is optionally replaced with a halogen, OR¹⁵, SR¹⁵, CN, N₃, NO₂, NR¹⁵R¹⁶, C(O)R¹⁵, C(S)R¹⁵, CO₂R¹⁵, C(O)SR¹⁵, C(O)NR¹⁵R¹⁶, C(S)NR¹⁵R¹⁶, NR¹⁵C(O)R¹⁶, NR¹⁵C(S)R¹⁶, NR¹⁵CO₂R¹⁶, NR¹⁵C(O)SR¹⁶, NR¹⁵C(O)NR¹⁶R¹⁷, or NR¹⁵C(S)NR¹⁶R¹⁷.

It another specific embodiment of the invention, when R¹ is an alkyl or an alkenyl radical (i.e., an alkyl or an alkenyl substituent), then it is a C₁-C₆ alkyl or, in the case when R¹ is an alkenyl, it is a C₂-C₆ alkenyl. When R¹ is a monocyclic substituent such as, for example, a cycloalkyl, a heterocycloalkyl, an aryl, or a heteroaryl, it can specifically comprise 4-7 members in the ring that defines the monocyclic skeleton. When R⁷, R⁸ or R⁹ is an unsubstituted alkyl, it can specifically be a C₁-C₆ unsubstituted alkyl; and when R⁷, R⁸ or R⁹ is an unsubstituted alkenyl, it can specifically be a C₂-C₆ unsubstituted alkenyl. The ring defined by R³ can specifically comprise 4-7 members or, in the case of polycyclics, each ring can comprise 4-7 members. When R³ is (CH₂)ₚR¹¹, the ring defined by R¹¹ can specifically comprise 4-7 members, or, in the case of polycyclics, each ring-can comprise 4-7 members. When either of R¹² or R¹³ is an unsubstituted alkyl, it can specifically be a C₁-C₆ unsubstituted alkyl, and when either of R¹² or R¹³ is an unsubstituted alkenyl, it is a C₂-C₆ unsubstituted alkyl. When R¹⁴ is a cycloalkyl, a heterocycloalkyl, an aryl, or a heteroaryl, the ring defined by R¹⁴ can specifically comprise 4-7 members, or, in the case of polycyclics, each ring can specifically comprise 4-7 members. When R⁶ is a cycloalkyl, a heterocycloalkyl, aryl, or a heteroaryl, the ring defined by R⁶ can specifically comprise 4-7 members, or, in the case of polycyclics, each ring can specifically comprise 4-7 members, and when R⁶ is substituted with a substituent that is an alkyl, an alkylthio, or an alkylamino, the substituent can specifically comprises from one to six carbon atoms, and when R⁶ is substituted with a substituent that is a cycloalkyl, a heterocycloalkyl, an aryl, or a heteroaryl, the ring defined by the substituent can specifically comprise 4-7 members or, in the case of polycyclics, each ring can specifically comprise 4-7 members.

In one embodiment of the invention, the method of preventing the emergence of resistance in accordance with the present invention includes administering a compound of Formula (I), wherein Q is C(O), R² is H, and W is C(O) or SO₂. In another embodiment of the invention, Q is C(O), R² is H, W is SO₂, and the stereochemical orientation of the asymmetric centers is represented by formula (IA) or (IB) below:

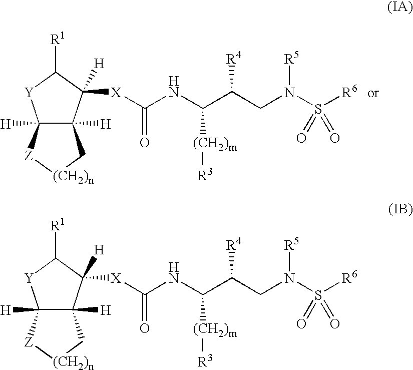

In another embodiment of the invention, R⁶ is a monocyclic substituent, (e.g. an aromatic ring), which can be a substituted benzene ring, as illustrated by the formula (IC) or (ID): 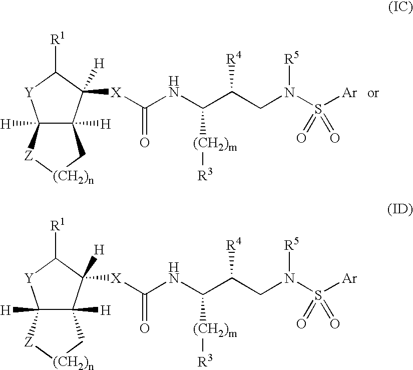
wherein Ar is a phenyl which is optionally substituted with a substituent selected from the group consisting of methyl, amino, hydroxy, methoxy, methylthio, hydroxymethyl, aminomethyl, and methoxymethyl.

In another embodiment of the invention, Y and Z are oxygen atoms, n is 2, the resulting bis-tetrahydrofuranyl ring system has the stereochemical orientations illustrated in Formulae (1C) and (ID) above, m is 1, and R³ is phenyl, in which case the compound is represented by the formula (IE) or (IF): 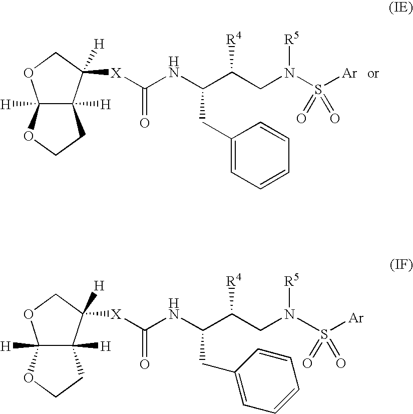
wherein Ar is a phenyl which is optionally substituted with a substituent selected from the group consisting of methyl, amino, hydroxy, methoxy, methylthio, hydroxymethyl, aminomethyl, and methoxymethyl. In another embodiment of the invention, when the compound is a compound of Formula (IE) or (IF), wherein at least one hydrogen atom on Ar is replaced with a substituent selected from the group consisting of methyl, amino, hydroxy, methoxy, methylthio, hydroxymethyl, and methoxymethyl, then X is an oxygen. In another embodiment of the invention, X is an oxygen and R⁵ is isobutyl. Suitable Ar substituents include phenyl groups that are substituted at the para position, the meta position, and/or the ortho position.

A resistance-inhibiting effective amount is an amount sufficient to produce an in vivo drug concentration or level in which the biochemical vitality of a mutant HIV is lower than the biochemical vitality of the HIV (predecessor) infecting the HIV-infected mammal. For example, a resistance-inhibiting effective amount is an amount sufficient to produce an in vivo drug concentration or level where the value for biochemical fitness is less than one, when determined by the ratio of the biochemical vitality of the mutant to the biochemical vitality of the predecessor. The compound can be administered to a wild-type HIV-infected mammal to prevent the emergence of first line resistance, or it can be administered to a mammal infected with a mutant-HIV to prevent the emergence of drug resistance due to further mutations.

The compound can be administered in the form of a pharmaceutical composition. The pharmaceutical composition can include a pharmaceutically acceptable carrier and a resistance-inhibiting effective amount of at least one of the aforesaid compound, alone or in combination with another antiretroviral compound such as, for example, a wild-type HIV protease inhibitor, a mutant HIV retroviral protease inhibitor, or a reverse transcriptase inhibitor. Generally, the pharmaceutical composition of the present invention comprises a resistance-inhibiting effective amount of at least one compound of Formula (I), as disclosed herein, and a pharmaceutically acceptable carrier.

In one embodiment of the invention, a pharmaceutical composition is administered that comprises a resistance-inhibiting effective amount of at least one compound of Formula (IA) or Formula (IB), or a pharmaceutically acceptable salt, prodrug, or ester thereof, and a pharmaceutically acceptable carrier. In a further embodiment of the invention, the pharmaceutical composition comprises a resistance-inhibiting effective amount of at least one compound of Formula (IC) or Formula (ID), or a pharmaceutically acceptable salt, prodrug, or ester thereof, and a pharmaceutically acceptable carrier. In yet another embodiment of the invention, the pharmaceutical composition comprises a resistance-inhibiting effective amount of at least one compound of Formula (IE) or Formula (IF), and pharmaceutically acceptable salts, prodrugs, and esters thereof, and a pharmaceutically acceptable carrier.

Pharmaceutically acceptable carriers are well-known to those of skill in the art. The choice of a carrier will be determined in part by the particular composition, as well as by the particular mode of administration. Accordingly, there are a wide variety of suitable formulations for administration in accordance the present invention.

The pharmaceutical composition may be administered in a form suitable for oral use such as, for example, tablets, troches, lozenges, aqueous or oily suspensions or solutions, dispersible powders or granules, emulsions, hard or soft capsules, syrups or elixirs. Compositions intended for oral use may be prepared according to any method known in the art form the manufacture of pharmaceutical compositions, and such compositions can contain one or more agents such as, for example, sweetening agents, flavoring agents, coloring agents, and preserving agents in order to provide a pharmaceutically elegant and/or palatable preparation. Tablets can contain the active ingredient in admixture with non-toxic pharmaceutically acceptable excipients which are suitable for manufacture of tablets. Such excipients can be, for example, inert diluents such as, for example, calcium carbonate, lactose, calcium phosphate or sodium phosphate; granulating and disintegrating agents such as, for example, maize starch or alginic acid; binding agents such as, for example, starch, gelatine or acacia, and lubricating agents such as, for example, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate alone or with a wax may be employed.

Formulations for oral use also can be presented as hard gelatin capsules wherein the active ingredient is mixed with an inert solid diluent, for example calcium carbonate, calcium phosphate or kaolin, or as soft gelatin capsules wherein the active ingredient is mixed with water or an oil medium, for example arachis oil, peanut oil, liquid paraffin or olive oil.

Aqueous suspensions typically contain the active materials in admixture with excipients suitable for the manufacture of aqueous suspensions. Such excipients are suspending agents, for example, sodium carboxymethyl cellulose, methylcellulose, hydroxypropylmethylcellulose, sodium alginate, polyvinylpyrrolidone, gum-tragacanth and gam acacia; dispersing or wetting agents may be a natural-occurring phosphatide, for example, lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation product-s of ethylene oxide with partial esters derived from fatty acids and hexitol anhydrides, for example polyoxyethylene sorbitan mono-oleate. The aqueous suspensions also can contain one or more preservatives, for example, ethyl or n-propyl p-hydroxy benzoate, one or more coloring agents, one or more flavoring agents and one or more sweetening agents such as, for example, sucrose or saccharin.

Oily suspensions may be formulated by suspending the active ingredient in a vegetable oil, for example arachis oil, olive oil, sesame oil or coconut oil, or in a mineral oil such as liquid paraffin. The oil suspensions may contain a thickening agent, for example beeswax, hard paraffin or cetyl alcohol. Sweetening agents, such as those set forth above, and flavoring agents may be added to provide a palatable oral preparation. These compositions can be preserved by the addition of an antioxidant such as, for example, ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, also may be present.

The pharmaceutical composition also can be administered in the form of oil-in-water emulsions. The oily phase can be a vegetable oil, for example, olive oil or arachis oils, or a mineral oil, for example liquid paraffin or mixtures of these. Suitable emulsifying agents may be naturally-occurring gums, for example gum acacia or gum tragacanth, naturally-occurring phosphatides, for example soya bean lecithin, and esters or partial esters derived from fatty acids and hexitol anhydrides, for example sorbitan mono-oleate, and condensation products of the said partial esters and ethylene oxide, for example polyoxyethylene sorbitan mono-oleate. The emulsions also can contain sweetening and flavoring agents.

The pharmaceutical composition also can be administered in the form of syrups and elixirs, which are typically formulated with sweetening agents such as, for example, glycerol, sorbitol or sucrose. “Such formulations also can contain a demulcent, a preservative and flavoring and coloring agents.

Further, the pharmaceutical composition can be administered in the form of a sterile injectable preparation, for example, as a sterile injectable aqueous or oleagenous suspension. Suitable suspensions for parenteral administration can be formulated according to the known art using those suitable dispersing or wetting agents and suspending agents which have been mentioned above. Formulations suitable for parenteral administration include, for example, aqueous and non-aqueous, isotonic sterile injection solutions, which can contain anti-oxidants, buffers, bacteriostates, and solutes that render the formulation isotonic with the blood of the intended recipient, and aqueous and non-aqueous sterile suspensions that can include suspending agents, solubilizers, thickening agents, stabilizers, and preservatives. The sterile injectable preparation can be a solution or a suspension in a non-toxic parenterally-acceptable diluent or solvent, for example, as a solution in water or 1,3-butanediol. Among the acceptable vehicles and solvents that can be employed, for example, are water, Ringer's solution and isotonic sodium-chloride solution. In addition, sterile, fixed oils are conventionally employed as a solvent or suspending medium. For this purpose any bland fixed oil can be employed including synthetic mono- or diglycerides. In addition, fatty acids such as, for example, oleic acid find use in the preparation of injectables.

Further, the compound can be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials include, for example, cocoa butter and polyethylene glycols. Formulations suitable for vaginal administration can, be presented as pessaries, tampons, creams, gels, pastes, and foams.

Formulations suitable for topical administration may be presented as creams, gels, pastes, or foams, containing, in addition to the active ingredient, such carriers as are known in the art to be appropriate.

The composition can be made into an aerosol formulation to be administered via inhalation. Such aerosol formulations can be placed into pressurized acceptable propellants, such as dichlorodifluoromethane, propane, nitrogen, and the like. They also can be formulated as pharmaceuticals for non-pressured preparations such as in a nebulizer or an atomizer.

The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Extemporaneous injection solutions and suspensions can be prepared from sterile powders, granules, and tablets of the kind previously described.

Any suitable dosage level can be employed in the pharmaceutical compositions of the present invention. The dose administered to an animal, particularly a human, in the context of the present invention should be sufficient to effect a prophylactic or therapeutic response in the animal over a reasonable time frame. The amount of active ingredient that can be combined with the carrier materials to produce a single dosage form will vary depending upon the host treated and the particular mode of administration. The size of the dose also will be determined by the existence, nature, and extent of any adverse side-effects that might accompany the administration of a particular composition. Suitable doses and dosage regimens for the prevention of drug resistance can be determined by comparisons to antiretroviral chemotherapeutic agents that are known to inhibit the proliferation of a retrovirus in an infected individual. The preferred dosage is the amount that results in the inhibition of the emergence of mutant drug-resistant retroviruses, particularly the emergence of multidrug-resistant retroviral HIV, without significant side effects. In proper doses and with suitable administration of certain compounds, a wide range of antiretroviral chemotherapeutic compositions are possible. A suitable dose includes a dose or dosage which would be insufficient to completely suppress the growth of a wild-type or predecessor virus, but would be sufficient to inhibit or effectively suppress the growth of a mutant.

In accordance with the present invention, the compound or composition can be administered in combination with other antiretroviral compounds such as, for example, ritonavir, amprenavir, saquinavir, indinavir, AZT, ddI, ddC, D4T, lamivudine, 3TC, and the like, as well as admixtures and combinations thereof, in a pharmaceutically acceptable carrier. The individual daily dosages for these combinations can range from about one-fifth of the minimally recommended clinical dosages to the maximum recommended levels for the entities when they are given singly

The present invention also provides a method of preventing the emergence of multidrug-resistant retroviruses in an HIV-infected mammal, which method comprises administering to the mammal a multidrug resistance-inhibiting effective amount of a compound of the present invention, so as to inhibit the emergence of a multidrug-resistant retrovirus in the mammal. The dose administered to an animal, particularly a human in the context of the present invention, should be sufficient to effect a therapeutic response in the animal over a reasonable time frame. The dose will be determined by the strength of the particular composition employed and the condition of the animal, as well as the body weight of the animal to be treated. The size of the dose will also be determined by the existence, nature, and extent of any adverse side-effects that might accompany the administration of a particular compound. Other factors which effect the specific dosage include, for example, bioavailability, metabolic profile, and the pharmacodynamics associated with the particular compound to be administered in a particular patient. One skilled in the art will recognize that the specific dosage level for any particular patient will depend upon a variety of factors including, for example, the activity of the specific compound employed, the age, body weight, general health, sex, diet, time of administration, route of administration, rate of excretion, drug combination, CD4 count, the potency of the active compound with respect to the particular mutant retroviral strain to be inhibited, and the severity of the symptoms presented prior to or during the course of therapy. What constitutes a resistance-inhibiting effective amount can be determined, in part, by use of one or more of the assays described herein, particularly the fitness assay of the present invention.

One skilled in the art will appreciate that suitable methods of administering compounds and pharmaceutical compositions are available, and, although more than one route can be used to administer a particular composition, a particular route can provide a more immediate and/or more effective reaction than another route.

Preparation of Compounds of the Invention

The compounds of the present invention can be synthesized by any suitable method known in the art. For example, suitable methods for preparing compounds of the invention are reported in U.S. patent application Ser. No. 11/030,632, which was published on 21 Jul. 2005 as United States Patent Application Publication Number US 2005/0158713.

It will be appreciated by a person of ordinary skill in the art that there are combinations of substituents, functional groups, R-groups, and the like, which are reactive under particular reaction conditions, and require the utilization of an appropriate protecting group or groups, which are known in the art, to ensure that the desired synthetic transformation will take place without the occurrence of undesired side reactions. For example, possible substituents at R⁵ (e.g., NH₂) can be competitive nucleophiles requiring the attachment of an appropriate protecting group thereon (e.g., benzyloxycarbonyl, tert-butoxycarbonyl) in order obtain proper selectivity in the ring opening of epoxide (i) with amine (ii).

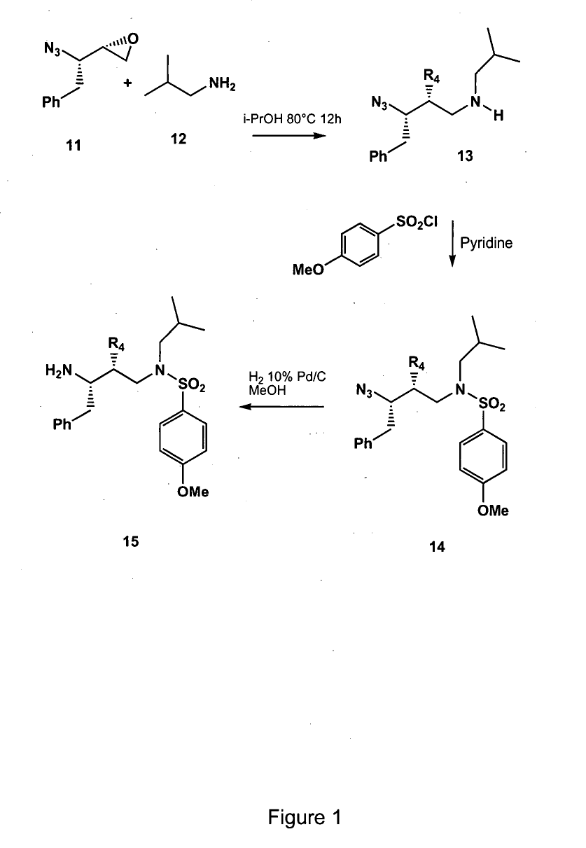

Together, FIGS. 1-3B illustrate the synthesis of compounds of the invention. With reference to FIG. 1, aminosulfonamide core 15 can be synthesized by initially providing azidoepoxide 11 and subjecting it to nucleophilic addition with amine 12 to give aminoalcohol 13, which is subsequently converted to sulfonamide 14 by reaction with 4-methoxybenzenesulfonyl chloride. The azide group of 14 is then reduced to provide aminosulfonamide 15, which can be used as a core for synthesizing numerous multidrug-resistant retroviral protease inhibitors of the present invention.

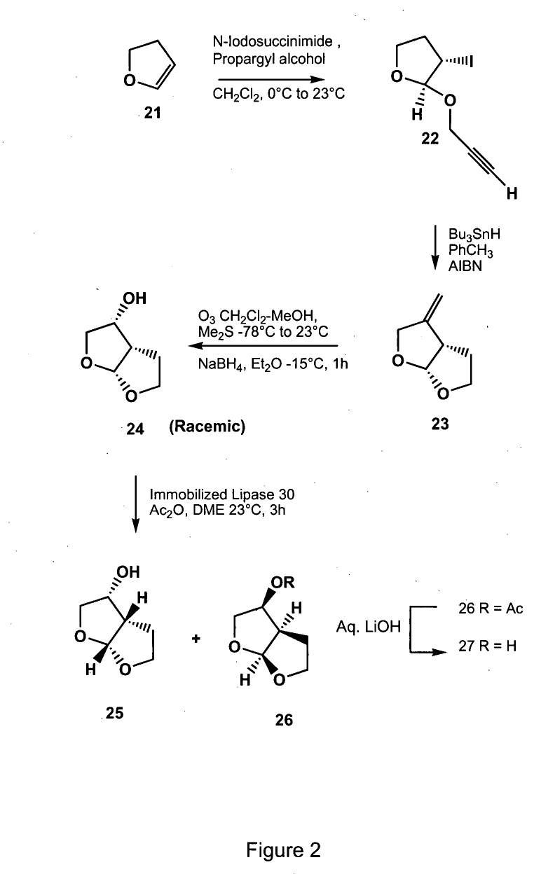

With reference to FIG. 2, dihydrofuran 21 is treated with N-iodosuccinimide in the presence of propargyl alcohol to give iodoether 22, which is cyclized to methylene-substituted bis-tetrahydrofuran 23. Ozonolysis of the exo-methylene residue of 23, followed by reduction, provides bicyclic racemic alcohol 24, which is resolved to give, separately, bicyclic alcohol 25 and its enantiomeric acetate ester 26, which ester group of 26 is subsequently hydrolyzed to afford enantiomer 27.

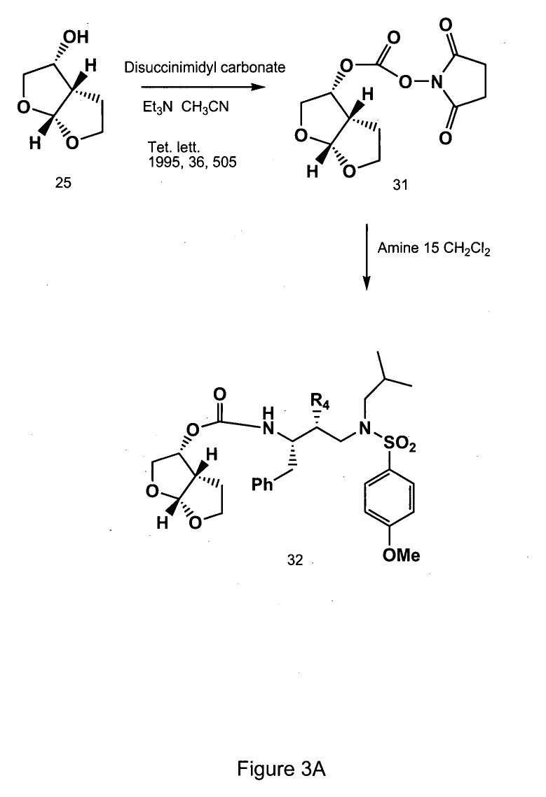

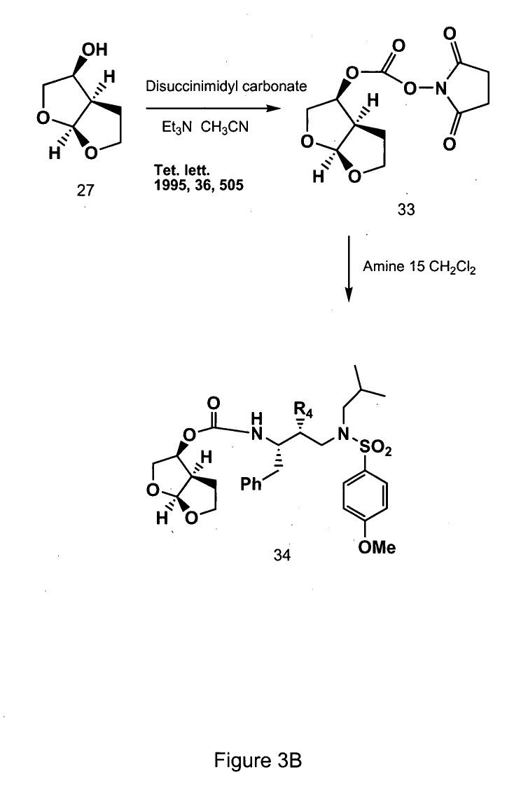

FIGS. 3A and 3B, illustrate the preparation of two compounds of the invention. With reference to FIG. 3A, compound 32 can be synthesized by coupling succinimidocarbonate 31 with aminosulfonamide 15. Succinimidocarbonate 31 can be prepared by reacting optically pure bicyclic alcohol 25 with disuccinimidyl carbonate in the presence of triethylamine. Inhibitor 34, which possesses the enantiomeric bis-tetrahydrofuranyl ligand (relative to inhibitor 32), can be prepared in the same fashion, except that the enantiomeric bicyclic alcohol 27 can be used instead of alcohol 25, as illustrated in FIG. 3B.

Biological Activity

The ability of a compound of the invention to inhibit the activity of one or more proteases or to prevent the development of drug resistance can be determined using any suitable method. For example, the compounds can be evaluated using the assay methods described in U.S. patent application Ser. No. 11/030,632, which was published on 21 Jul. 2005 as United States Patent Application Publication Number US 2005/0158713.

The liver stability of a compound of the invention can be determined using the S9 Assay described below.

### S9 Assay

Test compounds were transferred into 6 clusters of Marsh cluster tubes (2.5 μL of 0.2 mM in DMSO). An S9 suspension was prepared (6 mL) by dilution of stock S9 (In Vitro Technologies, MD) with 0.05M phosphate buffer, pH 7.4 to obtain an S9 protein concentration of 3.6 mg/mL. Three 8-tube clusters were filled with 600 μL/vial of dog, rat, human S9 suspensions, and 3 8-tube clusters were filled with 600 μL/vial of a NADPH/UDPGA solution (Sigma). The clusters were pre-heated for 5 minutes at 37° C., 250 μL of pre-heated S9 suspension media was transferred to compound tubes followed by addition of cofactors to the corresponding duplicate clusters to start the reaction. At 0, 5, 15, 30, 45 and 60 minutes, 25 μL of the reaction mixture was transferred to quench plates prefilled with Internal Standard/mobile Phase B (0.2% formic acid in 95% acetonitrile/5% water). The final composition of the reaction mixture was: 0.25 mL of 2 M compound, 3 mg S9 protein/mL, 1.25 mM NADPH, 2 mM UDPGA, 3.3 mM MgCl₂ in 0.05 M phosphate buffer, pH 7.4.

After quenching, 250 μL of water was added and the plates were centrifuged at 3K×G for 10 minutes. For analyses, 20 μL of the sample were injected into a Sciex 4000 Q-Trap LC/MS/MS (LOQ=0.001 μM). The column was 30×2 mm, 3 um Luna, maintained at room temperature. Mobile phase A was 0.2% formic acid in 1% ACN, mobile phase B was 0.2% formic acid in 95% ACN. Gradient was 0 to 90% B in 1.5 minutes with a total run time of 3 minutes. For some compounds mobile phase A was 20 mM ammonium acetate in 5% ACN and mobile phase B was 20 mM ammonium acetate in 80% ACN with a gradient of 0 to 100% B in 1.5 minutes with a total run time of 3 min. Data (analyte to IS area ratio) were plotted on a semilog scale and fitted using an exponential fit: C=Cₒ*exp^{(-Kt)}. Assuming the first order kinetics, the T_{1/2} and rate of metabolism were determined from the K values (T_{1/2}=Ln(2)/K and rate=amount of drug/mg protein×K=1000 pmol/mg×K).

The intrinsic hepatic clearance, Clint', was calculated from in vitro T_{1/2} data as described by Obach et al (Obach R S, Baxter J G, Liston T E, Silber B M, Jones B C, MacIntyre F, Rance D J, Wastall P. The prediction of human pharmacokinetic parameters from preclinical and in vitro metabolism data, J Pharmacol Exp Ther. 1997; 283 (1):46-58). Typically, compounds of the invention that were tested demonstrated a T_{1/2} of at least about 10 minutes against human S9. Some compounds demonstrated a T_{1/2} of at least about 100 minutes against human S9, while other compounds did not show significant degradation under the test conditions (i.e. the compounds were stable as tested).

The anti-HIV activity of a compound can be determined using the HIV Assay described below.

### HIV Assay

The Anti-HIV assay was carried out in a 96-well Clear Bottom Black Assay Plate (Costar # 3603) in 100 μL of culture medium, using the CellTiter-Glo™ Reagent (Promega # G7570) for signal detection. MT-2 cells (1.54×10⁴ cells) are infected with wild-type virus at an m.o.i. (multiplicity of infection, i.e. the ratio between the number of infectious viral particles and cells in an assay) of about 0.025, and grown in the presence of various drug concentrations (serial 5-fold dilutions) in 100 μL of RPMI medium containing 10% FBS, 2% glutamine, 1% HEPES and 1% penicillin/streptomycin for 5 days. At the end of the incubation period, 100 μL of CellTiter-Glo™ Reagent is added to each well in the Assay Plate and the chemiluminescence (in relative light units) is measured after 10 mins of incubation with the Wallac Victor² 1420 MultiLabel Counter. Representative compounds of the invention typically had an anti-HIV MT2 EC₅₀ of less than about 2 μM. Some compounds had an anti-HIV MT2 EC₅₀ of less than about 0.5 μM, while others had an anti-HIV MT2 EC₅₀ less than about 20 nM. Representative compounds of the invention were also tested against drug-resistant HIV strains. The compounds typically had an anti-HIV MT2 EC₅₀ of less than about 1 μM against the tested drug resistant strains.

The cytotoxicity of a compound can be determined using the Cytotoxicity Assay described below.

### Cytotoxicity Assay

### CC₅₀ Determination

The plate and reagents are the same as those described in the HIV Assay. Uninfected MT-2 cells (1.54.times.10.sup.4 cells) were grown in the presence of various drug concentrations (serial 2-fold dilutions) in 100 μl of RPMI medium containing 10% FBS, 2% glutamine, 1% HEPES and 1% penicillin/streptomycin for 5 days. At the end of the incubation period, 100 μL of CellTiter-Glo™. Reagent was added to each well in the assay plate and the chemiluminescence (in relative light units) was measured after 10 minutes of incubation with the Wallac Victor² 1420 MultiLabel Counter.

The invention will now be illustrated by the following non-limiting Examples.

### EXAMPLES

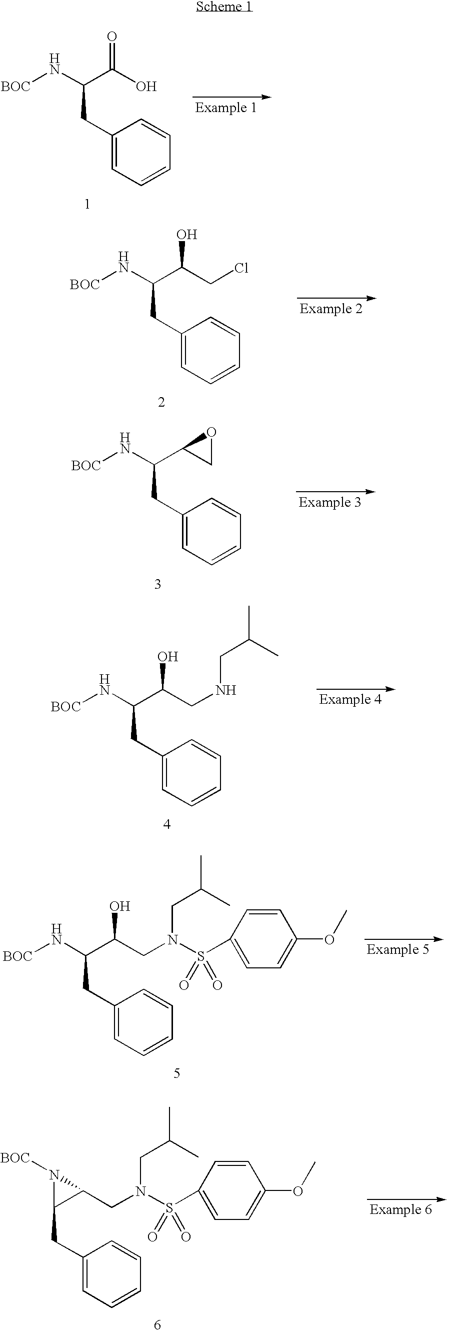

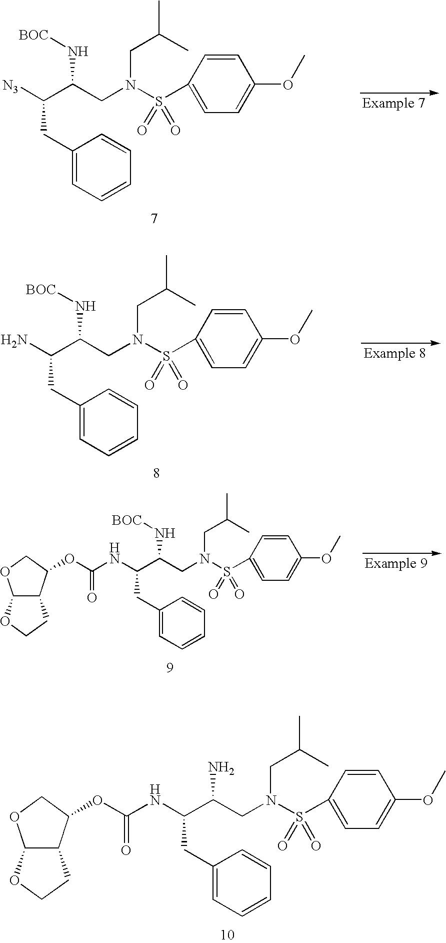

### Example 1

To a solution of N-Boc-D-phenylalanine 1 (5.0 g, 18.8 mmol) in THF (40 mL) at −20° C. was added isobutyl chloroformate (2.6 mL, 19.8 mmol) and 4-methylmorpholine (2.2 mL, 19.8 mmol). The reaction mixture was allowed to stir for 20 min, after which diazomethane (47.0 mmol) in Et₂O (100 mL) was added. The reaction mixture was allowed to warm to room temperature and stirred for 30 min. Nitrogen was bubbled into the reaction mixture for 1 h. The reaction mixture was partitioned between H₂O and Et₂O, and extracted with Et₂O. The organic phase was washed with H₂O, saturated NaHCO₃, and saturated NaCl. The organic phase was dried over Na₂SO₄, filtered, and evaporated under reduced pressure to give the crude diazoketone as a yellow solid (6.50 g).

To a solution of the crude diazoketone (18.8 mmol) in THF (100 mL) and Et₂O (100 mL) at 0° C. was added HCl in dioxane (5 mL, 19.8 mmol). The reaction mixture was allowed to stir for 2 h, after which the reaction mixture was evaporated under reduced pressure to give the crude chloroketone as a colorless solid (6.26 g).

To a solution of the crude chloroketone (18.8 mmol) in THF (120 mL) and H₂O (15 mL) at 0° C. was added sodium borohydride (1.49 g, 39.5 mmol). The reaction mixture was allowed to stir for 2 h, after which the reaction mixture was evaporated under reduced pressure. The reaction mixture was partitioned between H₂O and EtOAc, and extracted with EtOAc (500 mL). The organic phase was dried over Na₂SO₄, filtered, and evaporated under reduced pressure. The solid residue was recrystallized twice from EtOAc to give chlorohydrin 2 (2.91 g, 52% over 3 steps) as a colorless solid.

### Example 2

To a solution of chlorohydrin 2 (2.90 g, 9.67 mmol) in EtOH (150 mL) was added potassium hydroxide (650 mg, 11.6 mmol) in EtOH (20 mL). The reaction mixture was allowed to stir for 2 h, after which the reaction mixture was evaporated under reduced pressure. The reaction mixture was partitioned between saturated NH₄Cl and EtOAc. The organic phase was washed with saturated NH₄Cl. The organic phase was dried over Na₂SO₄, filtered, and evaporated under reduced pressure to give epoxide 3 (2.52 g, 99%) as a colorless solid.

### Example 3

To a solution of epoxide 3 (2.52 g, 9.57 mmol) in iPrOH (100 mL) at 80° C. was added isobutylamine (1.06 mL, 10.5 mmol). The reaction mixture was allowed to stir for 1 h, after which isobutylamine (1.06 mL, 10.5 mmol) was added again. The reaction mixture was allowed to stir for 1 h, after which the reaction mixture was evaporated under reduced pressure to give amine 4 (3.0 g, 93%) as a colorless solid.

### Example 4

To a solution of amine 4 (1.50 g, 4.46 mmol) in CH₂Cl₂ (40 mL) was added triethylamine (0.74 mL, 5.35 mmol) and 4-methoxybenzenesulfonyl chloride (1.09 g, 4.90 mmol). The reaction mixture was allowed to stir for 5 h, after which the reaction mixture was partitioned between saturated NH₄C₁ and CH₂Cl₂. The organic phase was washed with saturated NH₄Cl, dried over Na₂SO₄, filtered, and evaporated under reduced pressure. The crude product was chromatographed on silica gel (eluting 20-40% EtOAc/hexane) to give sulfonamide 5 (1.55 g, 69%) as a colorless foam.

### Example 5

To a solution of sulfonamide 5 (1.55 g, 3.06 mmol) in benzene (30 mL) was added triphenylphosphine (1.61 g, 6.12 mmol) and diisopropyl azodicarboxylate (1.2 mL, 6.12 mmol). The reaction mixture was allowed to stir for 16 h, after which the reaction mixture was partitioned between saturated NaHCO₃ and EtOAc, and extracted with EtOAc. The organic phase was dried over Na₂SO₄, filtered, and evaporated under reduced pressure. The crude product was chromatographed twice on silica gel (eluting 10-40% EtOAc/hexane and 0-10% EtOAc/CH₂Cl₂) to give crude aziridine 6 as a colorless oil.

### Example 6

To a solution of crude aziridine 6 (3.06 mmol) in iPrOH (30 mL) at 60° C. was added azidotrimethylsilane (0.80 mL, 6.12 mmol). The reaction mixture was allowed to stir for 6 h, after which azidotrimethylsilane (0.80 mL, 6.12 mmol) was added. The reaction mixture was allowed to stir for 16 h, after which the reaction mixture was evaporated under reduced pressure. The crude product was chromatographed on silica gel (eluting 10-30% EtOAc/hexane) to give azide 7 (1.12 g, 69% over 2 steps) as a colorless solid.

### Example 7

To a solution of azide 7 (1.01 g, 1.90 mmol) in EtOH (20 mL) and EtOAc (20 mL) was added 10% palladium on carbon (101 mg) and fitted with a balloon filled with hydrogen. The reaction mixture was allowed to stir for 16 h, after which the reaction mixture was filtered through a pad of Celite and rinsed with MeOH and EtOAc. The crude product was chromatographed on silica gel (eluting 2-8% MeOH/CH₂Cl₂) to give amine 8 (552 mg, 57%) as a colorless oil.

### Example 8

To a solution of amine 8 (297 mg, 0.587 mmol) in CH₃CN (5 mL) was added bisfuran-4-nitrophenyl carbonate (260 mg, 0.881 mmol), diisopropylethylamine (0.20 mL, 1.17 mmol), and 4-dimethylaminopyridine (0.7 mg, 0.587 μmol). The reaction mixture was allowed to stir for 3 h, after which CH₂Cl₂ (5 mL) was added to partially dissolve the precipitate. The reaction mixture was allowed to stir for 16 h, after which the reaction mixture was evaporated under reduced pressure. The residue was partitioned between saturated NaHCO₃ and EtOAc, and extracted with EtOAc. The organic phase was dried over Na₂SO₄, filtered, and evaporated under reduced pressure. The crude product was chromatographed on silica gel (eluting 3-4% MeOH/CH₂Cl₂) and washed with 1M K₂CO₃ to give crude carbamate 9 (295 mg, 76%) as a colorless solid.

### Example 9

To a solution of carbamate 9 (296 mg, 0.446 mmol) in CH₂Cl₂ (8 mL) at 0° C. was added trifluoroacetic acid (2 mL). The reaction mixture was allowed to stir for 1 h, after which the reaction mixture was partitioned between saturated NaHCO₃ and CH₂Cl₂, and extracted with CH₂Cl₂. The organic phase was dried over Na₂SO₄, filtered, and evaporated under reduced pressure. The residue was partitioned between 1M K₂CO₃ and EtOAc and washed with 1M K₂CO₃ to give amine 10 (245 mg, 98%) as a pale yellow solid. M/Z 562.2 (M+1).

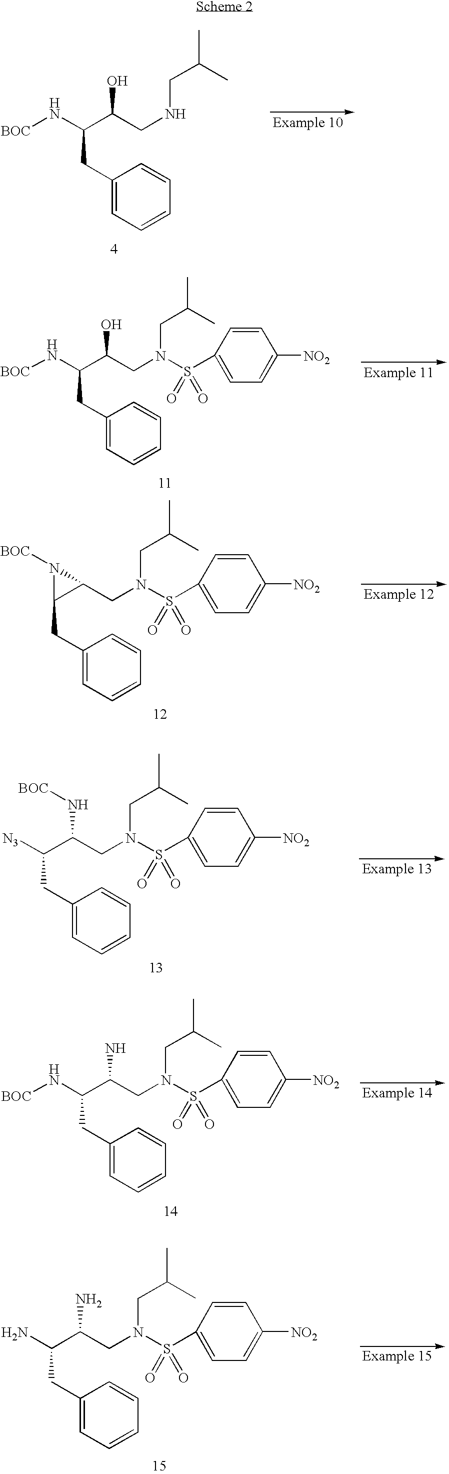

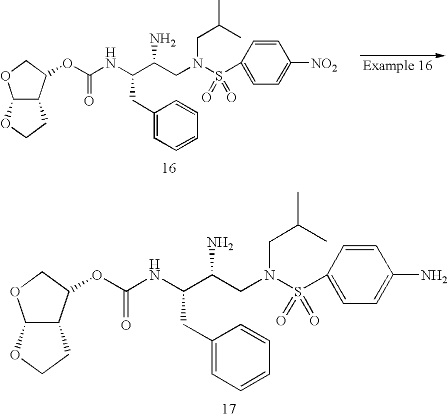

### Example 10

The synthesis of intermediate 11 followed the procedure in Example 4, except that 4-methoxybenzenesulfonyl chloride was replaced with 4-nitrobenzenesulfonyl chloride.

### Example 11

The synthesis of intermediate 12 followed the procedure in Example 5.

### Example 12

The synthesis of intermediate 13 followed the procedure in Example 6.

### Example 13

To a solution of azide 13 (114 mg, 0.208 mmol) in THF (2 mL) was added triphenylphosphine (60 mg, 0.229 mmol) and H₂O (0.2 mL). The reaction mixture was allowed to stir for 3 d, after which the reaction mixture was partitioned between saturated NaHCO₃ and EtOAc, and extracted with EtOAc. The organic phase was dried over Na₂SO₄, filtered, and evaporated under reduced pressure. The crude product was chromatographed on silica gel (eluting 20-50% EtOAc/hexane) to give amine 14 (69 mg, 64%) as a colorless oil.

### Example 14

To a solution of amine 14 (61 mg, 0.117 mmol) in CH₂Cl₂ (1.8 mL) was added trifluoroacetic acid (0.2 mL). The reaction mixture was allowed to stir for 1 h, after which the reaction mixture was evaporated under reduced pressure to give the crude diamine 15 (79 mg) as a pale yellow foam.

### Example 15

To a solution of crude diamine 15 (0.117 mmol) in CH₂Cl₂ (2 mL) was added bisfuran-4-nitrophenyl carbonate (52 mg, 0.176 mmol) and diisopropylethylamine (0.04 mL, 0.234 mmol). The reaction mixture was allowed to stir for 16 h, after which the reaction mixture was evaporated under reduced pressure. The residue was partitioned between saturated NaHCO₃ and EtOAc, and extracted with EtOAc. The organic phase was dried over Na₂SO₄, filtered, and evaporated under reduced pressure. The crude product was chromatographed on preparative thin layer chromatography (eluting 5% MeOH/CH₂Cl₂) and washed with 1M K₂CO₃ to give crude carbamate 16 (49 mg, 73%) as a colorless oil.

### Example 16

To a solution of carbamate 16 (49 mg, 0.085 mmol) in EtOH (1 mL) and EtOAc (0.5 mL) was added 10% palladium on carbon (10 mg) and fitted with a balloon filled with hydrogen. The reaction mixture was allowed to stir for 2 d, after which the reaction mixture was filtered through a pad of Celite and rinsed with MeOH and EtOAc. The crude product was chromatographed on preparative thin layer chromatography (eluting 5% MeOH/CH₂Cl₂) to give amine 17 (27 mg, 58%) as a colorless solid. M/Z 547.2 (M+1).

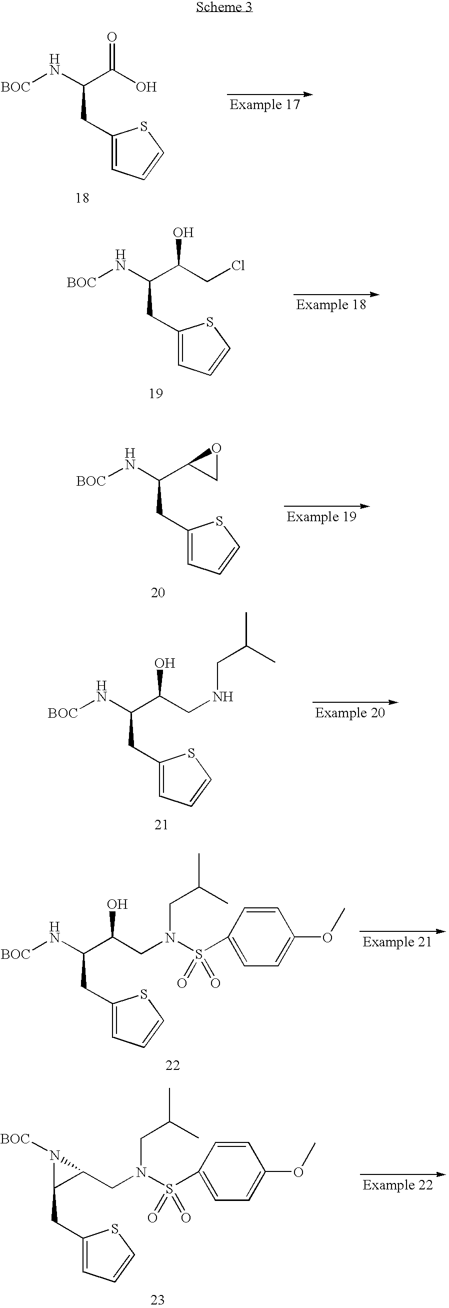

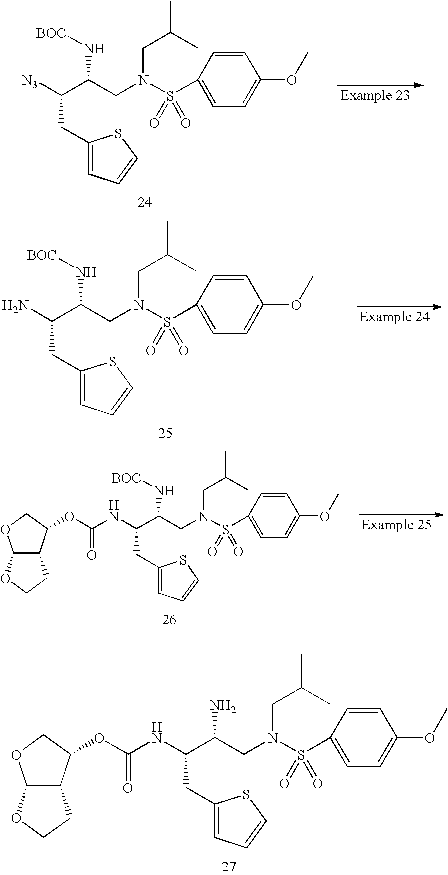

### Example 17

The synthesis of intermediate 19 followed the procedure in Example 1, except that N-Boc-D-phenylalanine was replaced with N-Boc-D-2-thienylalanine.

### Example 18

The synthesis of intermediate 20 followed the procedure in Example 2.

### Example 19

The synthesis of intermediate 21 followed the procedure in Example 3.

### Example 20

The synthesis of intermediate 22 followed the procedure in Example 4.

### Example 21

The synthesis of intermediate 23 followed the procedure in Example 5.

### Example 22

The synthesis of intermediate 24 followed the procedure in Example 6.

### Example 23

The synthesis of intermediate 25 followed the procedure in Example 7.

### Example 24

The synthesis of intermediate 26 followed the procedure in Example 8.

### Example 25

The synthesis of intermediate 27 followed the procedure in Example 9. M/Z=568.1 (M+1).

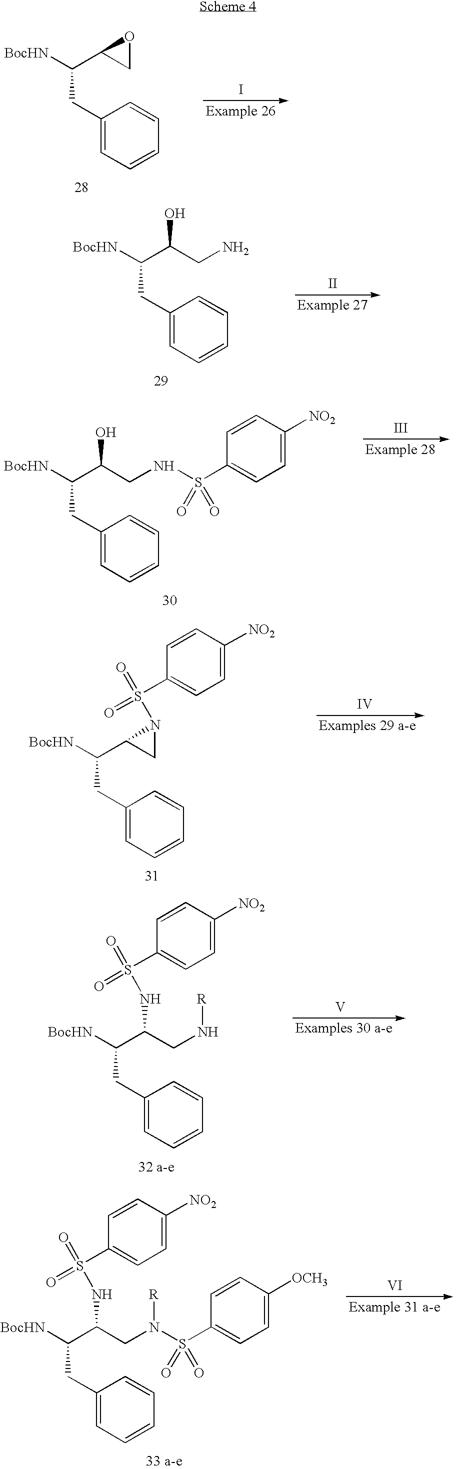

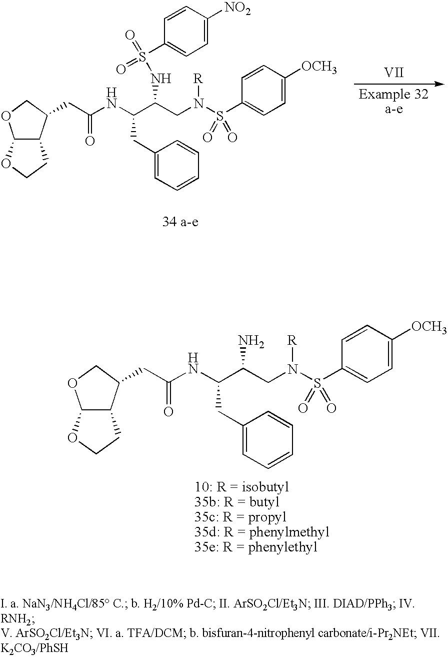

### Example 26

To a solution of epoxide 28 (4.0 g, 15.2 mmol) in EtOH (48 mL) and water (6 ml) were added ammonium chloride (1.62 g, 30.4 mmol) and sodium azide (2.0 g, 30.4 mmol). The reaction mixture was heated at 85° C. for 16 h. The mixture was cooled to 25° C. and the solvents were evaporated under reduced pressure. The reaction mixture was partitioned between water and EtOAc. The organic phase was washed with water and brine, and dried over Na₂SO₄. Evaporation under reduced pressure gave a white solid (4.8 g).

The mixture of above solid (1.0 g) and 10% Pd—C (100 mg) in isopropanol was hydrogenated for 2 hours. Celite (1 g) was added and the mixture was stirred for 5 minutes. Filtration and evaporation gave amino alcohol 29 (950 mg).

### Example 27

To a solution of amino alcohol 29 (560 mg, 2 mmol) in dichloromethane was added 4-nitrobenzenesulfonyl chloride (443 mg, 2 mmol), followed by triethylamine (0.56 ml, 4 mmol). The mixture was stirred for 12 hours, and diluted with ethyl acetate. The organic phase was washed with water and brine, was dried with sodium sulfate. Purification by flash column chromatography (hexane/EtOAc=1/1) gave compound 30 (790 mg).

### Example 28

To a solution of compound 30 (650 mg, 1.4 mmol) and triphenylphosphine (470 mg, 1.7 mmol) in THF at 0° C. was added diisopropyl azodicarboxylate (0.350 ml, 1.7 mmol) dropwise. The mixture was stirred at 0° C. for 2 hours and at 25° C. for 12 hours. Solvent was evaporated. Purification by flash column chromatography (hexane/EtOAc=4/1) gave compound 31 (210 mg).

### Example 29a

To a solution of compound 31 (34 mg, 0.07 mmol) in dichloromethane (1 ml) was added isobutylamine (76 μL, 0.7 mmol). The mixture was stirred at 25° C. for 2 hours. Evaporation gave compound 32a (39 mg).

### Example 30a

To a solution of amine 32a (39 mg, 0.07 mmol) in CH₂Cl₂ (0.5 mL) was added triethylamine (42 μL, 0.3 mmol) and 4-methoxybenzenesulfonyl chloride (18 mg, 0.08 mmol). The reaction mixture was stirred for 24 h, and was diluted with ethyl acetate. The organic phase was washed with saturated Na₂CO₃, water, and brine, and was dried over Na₂SO₄. The crude mixture was purified by flash column chromatography (hexanes/EtOAc=3.5/1) to give compound 33a (25 mg).

### Example 31a

The mixture of compound 33a (25 mg) in dichloromethane (1.5 ml) and trifluoroacetic acid (0.5 ml) was stirred for 1 hour. Solvent and reagent were removed under reduced pressure. The mixture was diluted with Ethyl acetate, and washed with saturated Na₂CO₃, water, and brine, and dried over Na₂SO₄. Concentration gave a brown solid.

To a solution of above solid in acetonitrile (0.5 ml) were added bisfuran-4-nitrophenyl carbonate (9 mg, 0.03 mmol), diisopropylethylamine (10 μL, 0.06 mmol), and 4-dimethylaminopyridine (1 mg). The mixture was stirred for 12 hours. Solvent and reagent were removed under reduced pressure. Purification by flash column chromatography (hexanes/EtOAc=1/1) gave compound 34a (20 mg).

### Example 32a

To a solution of compound 7a (13 mg) in acetonitrile (1 ml) was added DMSO (5 μL), followed by K₂CO₃ (70 mg) and thiophenol (44 μL). The mixture was heated at 50° C. for 2 hours. The mixture was diluted with water and extracted with ethyl acetate. The organic phase was washed with water and brine, and dried over Na₂SO₄. The crude product was purified by flash column chromatography (hexanes/EtOAc=1/1 to 100% EtOAc) to give compound 10 (8 mg). m/z: 562.2 (M+1), 584.3 (M+Na).

### Example 32b

Compound 35b (23 mg) was synthesized following the procedures for 35a. m/z: 562.2 (M+1), 584.3 (M+Na).

### Example 32c

Compound 35c (13 mg) was synthesized following the procedures for 35a. m/z: 548.2 (M+1), 570.3 (M+Na).

### Example 32d

Compound 35d (30 mg) was synthesized following the procedures for 35a. m/z: 596.1 (M+1), 618.2 (M+Na).

### Example 32e

Compound 35e (52 mg) was synthesized following the procedures for 35a. m/z: 610.2 (M+1); 632.2 (M+Na).

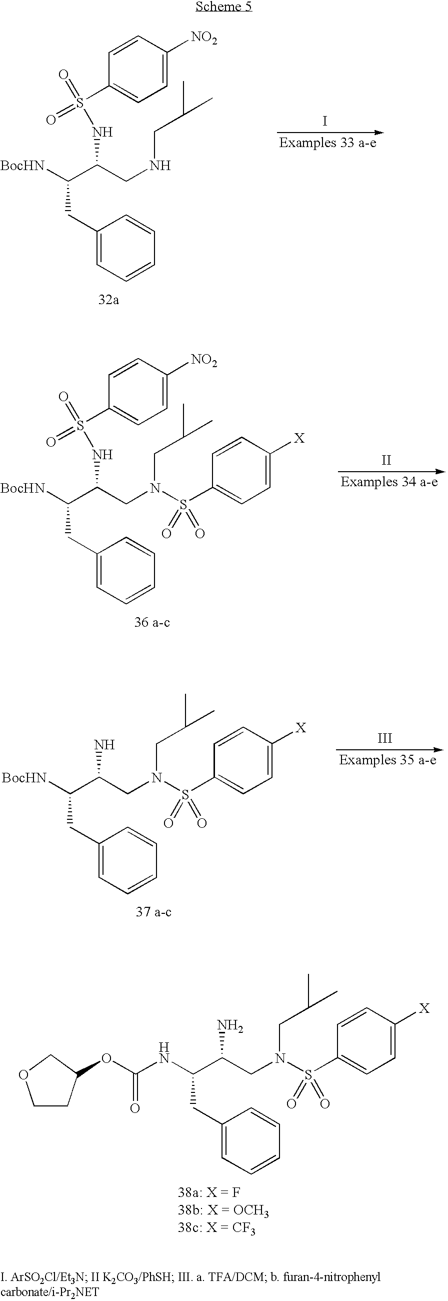

### Example 33a

The synthesis of intermediate 36a (50 mg) followed the procedure in Example 30a by using 4-Fluorobenzenesulfonyl chloride.

### Example 34a

The synthesis of intermediate 37a (25 mg) followed the procedure in Example 31a.

### Example 35a

The synthesis of compound 38a (3 mg) follows the procedure in Example 32a. m/z: 508.2 (M+1), 530.2 (M+Na).

### Example 35b

The synthesis of compound 38b (12 mg) followed the procedures for compound 38a. m/z: 520.2 (M+1).

### Example 35c

The synthesis of compound 38c (20 mg, 80% yield) followed the procedures for compound 38a. m/z: 558.2 (M+1).

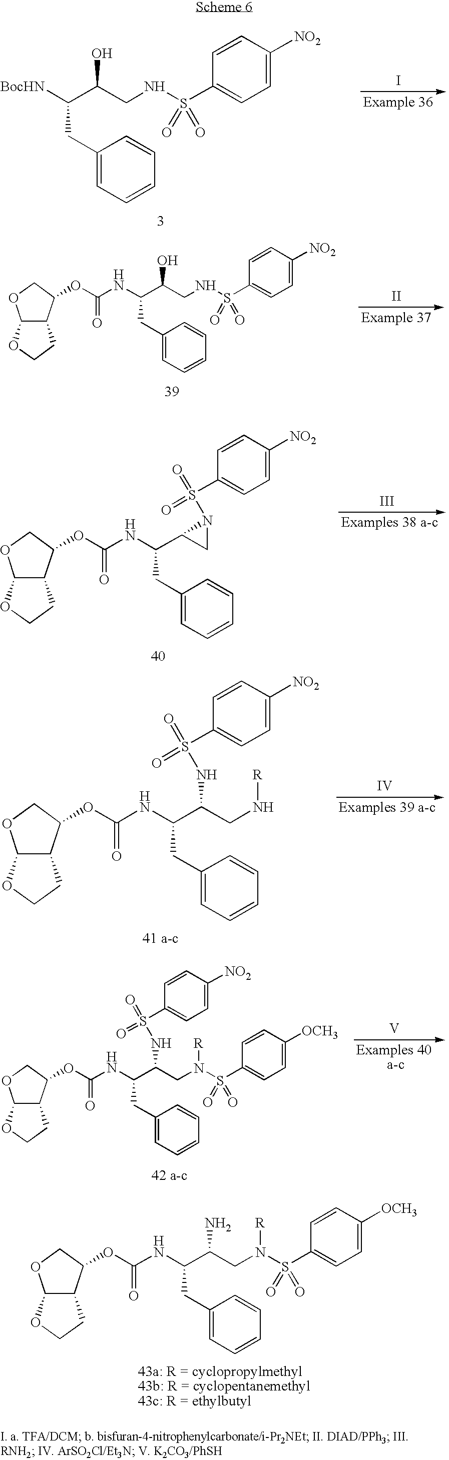

### Example 36

Compound 39 (900 mg) was synthesized following the procedure for compound 34a.

### Example 37

Compound 40 was synthesized following the procedure for compound 31.

### Example 38a

Compound 41a was synthesized following the procedure for compound 32a.

### Example 39a

Compound 42a was synthesized following the procedure for compound 33a.

### Example 40a

Compound 43a was synthesized following the procedure for compound 35a. m/z: 560.1 (M+1).

### Example 40b

Compound 43b was synthesized following the procedures for compound 43a. m/z: 588.2 (M+1).

### Example 40c

Compound 43c was synthesized following the procedures for compound 43a. m/z: 590.2 (M+1).

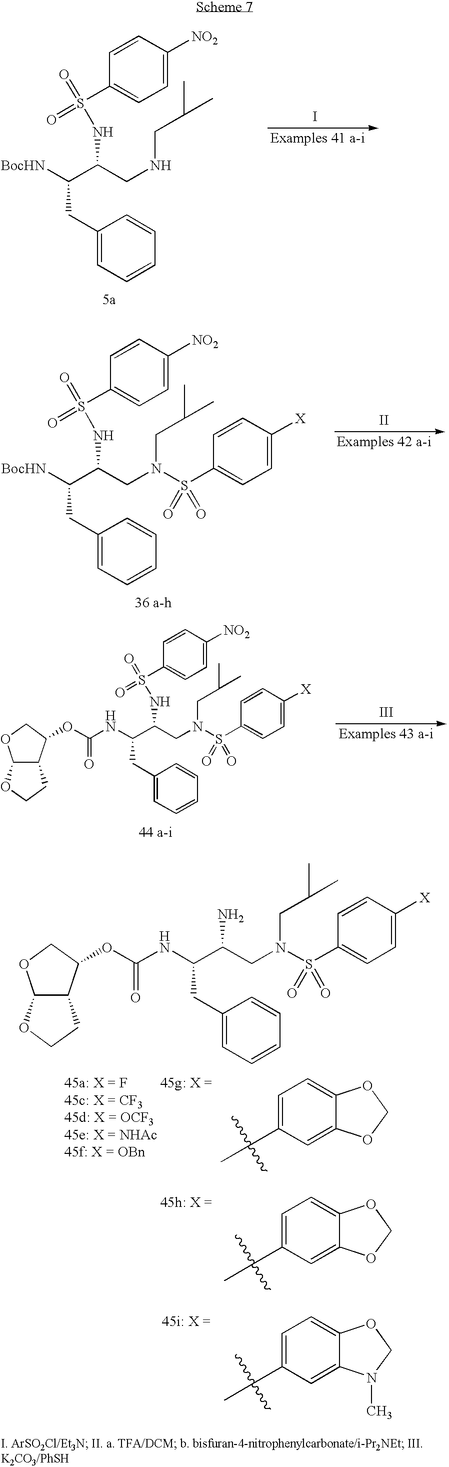

### Examples 41a-i

Compounds 36d-i were synthesized following the procedure for compound 36a using the appropriately substituted phenylsulfonyl chlorides as defined in compounds 45d-i.

### Examples 42a-i

Compounds 44a-i were synthesized following the procedure for compound 34a.

### Examples 43a-i

Compounds 45a-i were synthesized following the procedure for compound 35a. 45a m/z=550.1 45d m/z=616.1; 45e m/z=589.2; 45f m/z=638.2; 45g m/z=598.1; 45h m/z=590.2; and 45i m/z=633.2.

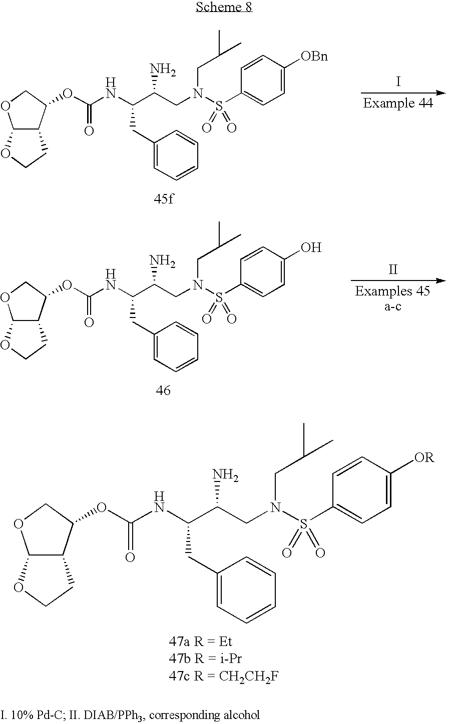

### Example 44

Compound 46 was prepared by dissolving compound 45f (90 mg) in 3 ml EtOH and 3 ml EtOAc, adding 20 mg 10% Pd/C, and stirring under a hydrogen atmosphere for 2 hours. The reaction mixture was filtered through Celite, concentrated and evaporated to yield 80 mg of compound 46. m/z=548.3.

### Example 45a

Compound 47a was prepared by dissolving 20 mg of compound 46 on 1 ml of THF, to which 48 mg of triphenylphosphine, 3.4 mg ethanol and lastly 42 mg Di-tert-butyl azodicarboxylate were added. The reaction stirred overnight and was purified by HPLC, then silica gel chromatography yielding 11 mg of compound 47a. m/z=576.2.

### Example 45b

Compound 47b was synthesized following the procedure for compound 47a using isopropanol in place of ethanol. m/z 590.2

### Example 45c

Compound 47c was synthesized following the procedure for compound 47a using 2-fluoroethanol in place of ethanol. m/z=594.1.

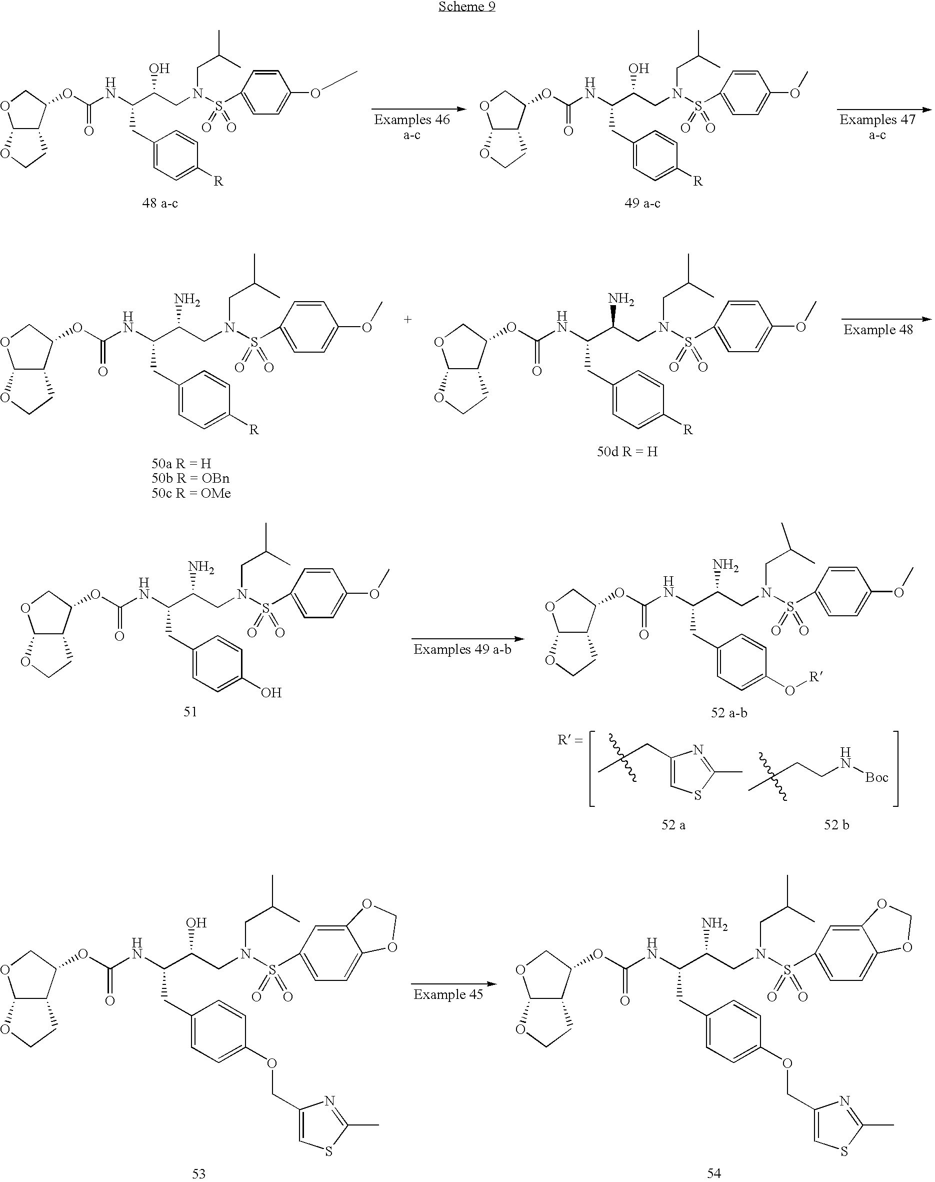

### Example 46a

To a solution of 48a (1.5 mmol) in DCM (15 mL) at 20° C. was added Dess-Martin periodinane (1.8 mmol). The reaction mixture was allowed to stir for 5 min, then was chromatographed on silica gel (eluting 50-100% EtOAc/hexane) to give 49a (1.425 mmol, 95%) as a white foam.

### Examples 46b-c

Compounds 49b and 49c were prepared analogously to compound 49a.

### Example 47a

To a solution of 49a (1.00 mmol) in methanol (60 mL) at 20° C. was added ammonium acetate (100 mmol), acetic acid (60.0 mmol), lithium chloride (15.0 mmol) and sodium cyanoborohydride (15.0 mmol). The reaction mixture was allowed to stir for 20 hours. The reaction mixture was concentrated and partitioned between saturated NaHCO₃ and EtOAc, and extracted with EtOAc. The organic phase was washed with H₂O and dried over Na₂SO₄, filtered, and evaporated under reduced pressure. The crude product was chromatographed on silica gel (eluting 0-10% MeOH/DCM) to give the pure isomer (lower Rf on TLC) of 50a (25%) as white foam, m/z=562.2 and pure isomer (higher Rf on TLC) of 50d (24%) as white foam, m/z=562.2.

### Examples 47b-c

Compounds 50b and 50c were prepared analogously to compound 50a.

### Example 48

To a solution of 50b (100 mg, 0.15 mmol) in EtOH (1.5 mL) and EtOAc (1.5 mL) was added acetic acid (13.5 mg, 0.225 mmol), then was added 10% palladium on carbon (15 mg) and fitted with a balloon filled with hydrogen. The reaction mixture was allowed to stir for 4 h, after which the reaction mixture was filtered through a pad of Celite and rinsed with EtOH and EtOAc. The organic phase was concentrated and partitioned between saturated NaHCO₃ and EtOAc, and extracted with EtOAc. The organic phase was washed with H₂O and dried over Na₂SO₄, filtered, and evaporated under reduced pressure. The crude product was chromatographed on silica gel (eluting 0-12% MeOH/DCM) to give 51 (80%) as clear film.

### Example 49a

To a solution of 51 (0.315 mmol) in THF (30 mL) was added 2-methyl-4-hydroxymethylthiazole (0.630 mmol), triphenylphosphine (0.945 mmol) and di-t-butyl azodicarboxylate (0.945 mmol). The reaction mixture was allowed to stir for 16 h, after which the reaction mixture was directly chromatographed on silica gel (eluting 0-10% MeOH/DCM) to give 52a (70%) as colorless film.

### Example 49b

Compound 52b was made analogously to compound 52a, using N-Boc-aminoethanol instead of the 2-methyl-4-hydroxymethylthiazole.

### Example 50

Compound 54 was made analogously to compound 50a, following the general procedures of Examples 46a and 47a.

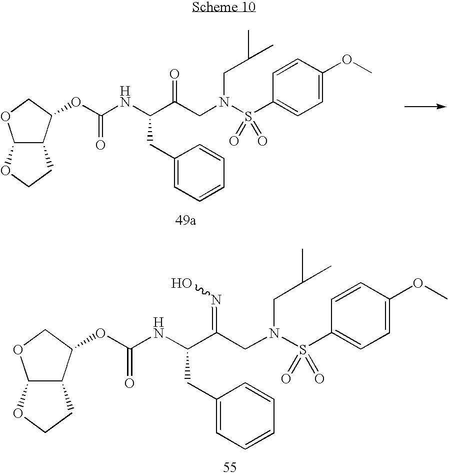

### Example 51

Compound 55 was prepared by dissolving 134 mg of compound 49a on 1 ml of pyridine, to which 18.5 mg of ammonium hydroxide hydrochloride were added. The reaction stirred 10 days and the reaction was concentrated, and then was purified by HPLC, yielding 120 mg of compound 23. m/z=598.2.

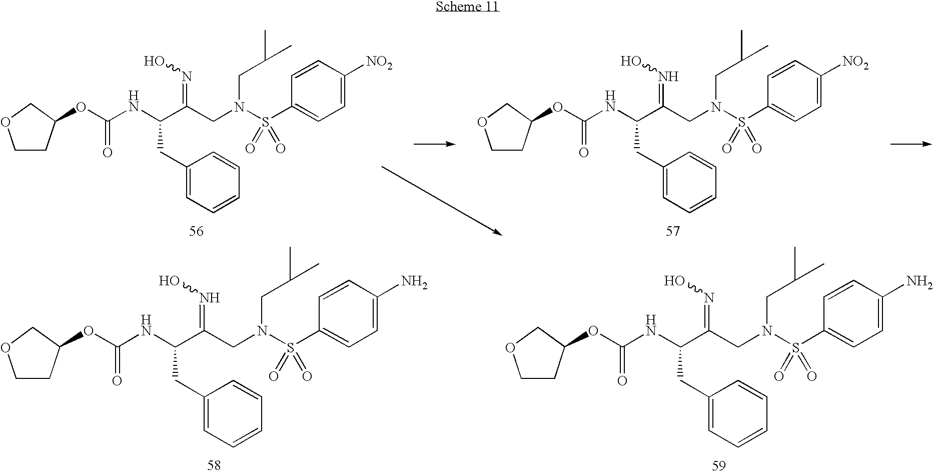

### Example 52

Compound 56 was prepared analogously to compound 55.

### Example 53

Compound 56 was dissolved in 0.2 mL of ethyl acetate and 0.2 mL of THF, to which was added dropwise 31 μL of 8M solution of borane-pyridine complex followed by 12 equivalents of 4M HCl in dioxane. The reaction stirred overnight, and was poured into saturated NaHCO₃, extracted with EtOAc, washed with brine, dried with Na₂SO₄, concentrated to provide compound 57 that was used directly in the next step.

### Example 54

Crude compound 57 (21 mg) was dissolved in 0.2 mL of acetic acid, to which was added 30 mg of zinc dust. The reaction stirred overnight, was filtered, concentrated and purified by HPLC to yield compound 58 (3.2 mgs) as the trifluoroacetic acid salt. m/z=521.2

### Example 55

Crude compound 56 (30 mg) was dissolved in 0.5 mL of acetic acid, to which was added 45 mg of zinc dust. The reaction stirred for 3 hours, was filtered, the remaining powder was washed with methanol and ethyl acetate, the combined organics were concentrated and the compound was purified by HPLC to yield 22.8 mg of compound 59. m/z 541.2 (M+Na).

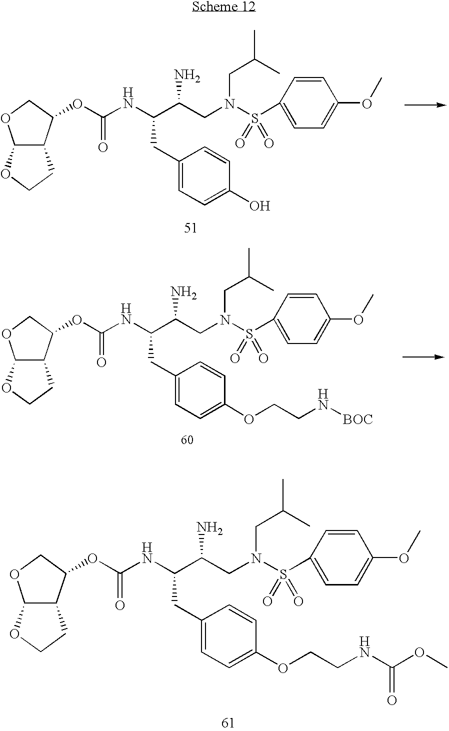

### Example 56

To a solution of compound 51 (0.315 mmol) in THF (30 mL) were added R′—OH (0.630 mmol), triphenylphosphine (0.945 mmol) and di-t-butyl azodicarboxylate (0.945 mmol). The reaction mixture was allowed to stir for 16 h, after which the reaction mixture was directly chromatographed on silica gel (eluting 0-10% MeOH/DCM) to give 60 (70%) as colorless film.

### Example 57

To a solution of compound 60 (0.245 mmol) in DCM (2 mL) were added TFA (0.4 mL) at 0° C. The reaction mixture was allowed to stir for 1 h the at 0° C. and 30 min at room temperature, after which Toluene (5 mL) was added then concentrated under 5° C. The compound prepared was dissolved in THF (4 mL) and sat. sodium bicarbonate solution (8 mL) was added to it. To the mixture was added 1.05 equiv. of methyl chloroformate in THF (4 mL). The reaction mixture was allowed to stir for 20 min. The reaction mixture was partitioned between brine and EtOAc, and extracted with EtOAc. The organic phase was washed with H₂O and dried over Na₂SO₄ and evaporated under reduced pressure. The crude product was chromatographed on silica gel (eluting 0-10% MeOH/DCM) and purified with prep HPLC to afford compound 61 (33%) as white solid. m/z 679.2

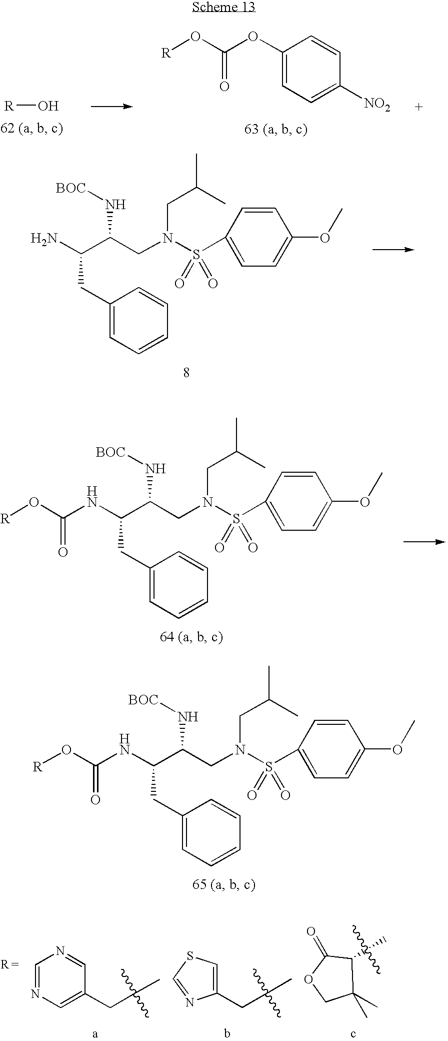

### Example 58

To a solution of 62a (266 mg, 2.42 mmol) in DCM (30 mL) were added triethylamine (0.51 mL, 3.63 mmol) and bis(4-nitrophenyl)carbonate (809 mg, 2.66 mmol). The reaction mixture was allowed to stir for 30 min, after which the reaction mixture was directly chromatographed on silica gel (eluting 40-100% EtOAc/hexane). The crude compound was partitioned with EtOAc and sat. potassium carbonate solution, extracted with EtOAc, and washed with water (3×) to give 63a (121 mg, 18%) as white solid.

Using a similar procedure, compound 63b was prepared in 91% yield.

Using a similar procedure, compound 63c was prepared

### Example 59

To a solution of amine 8 (25 mg, 0.049 mmol) in CH₃CN (2 mL) was added 63a (20 mg, 0.074 mmol) and diisopropylethylamine (0.099 mL, 0.099 mmol). The reaction mixture was allowed to stir for 18 h, after which the reaction mixture was evaporated under reduced pressure. The residue was partitioned between saturated NaHCO₃ and EtOAc, and extracted with EtOAc (2×) and CH₂Cl₂ (2×). The organic phase was dried over Na₂SO₄, filtered, and evaporated under reduced pressure. The crude product was chromatographed on preparative thin layer chromotography (eluting 7% MeOH/CH₂Cl₂) to give carbamate 64a (24 mg, 76%) as a colorless solid.

Using a similar procedure, compound 64b was prepared in 88% yield.

Using a similar procedure, compound 64c was prepared and used directly in the next Example.

### Example 60

To a solution of carbamate 64a (24 mg, 0.0374 mmol) in CH₂Cl₂ (1.6 mL) at 0° C. was added trifluoroacetic acid (0.4 mL). The reaction mixture was allowed to stir for 1 h, after which the reaction mixture was partitioned between saturated NaHCO₃ and CH₂Cl₂, and extracted with CH₂Cl₂. The organic phase was dried over Na₂SO₄, filtered, and evaporated under reduced pressure. The residue was partitioned between 1M K₂CO₃ and EtOAc and washed with 1M K₂CO₃ to give amine 65a (24 mg, 76%) as a pale yellow solid.

Using a similar procedure, compound 65b was prepared in 84% yield.

Using a similar procedure, compound 65c was prepared in 36% yield from compound 63c.

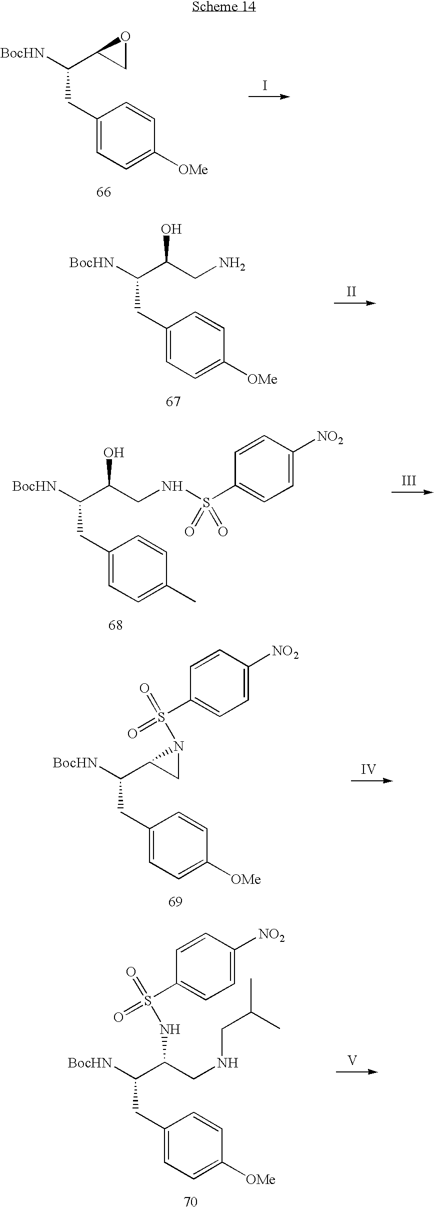

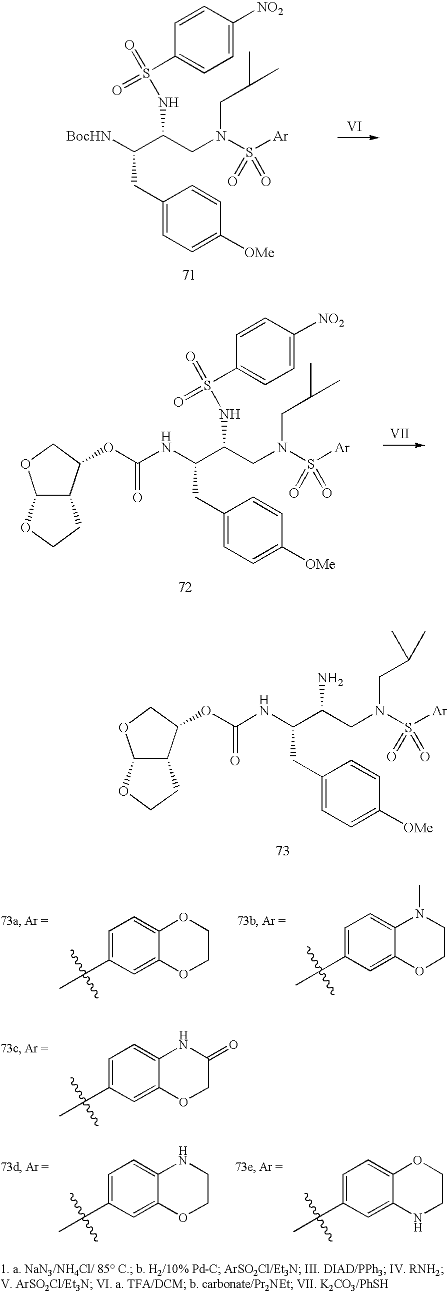

### Example 61a

Compound 73a (90 mg) was prepared following the procedure for compound 45a. m/z: 620.2 (M+1), 642.2 (M+Na).

### Example 61b

Compound 73b was prepared following the procedure for compound 45a.

### Example 61c

Compound 73c was prepared following the procedure for compound 45a. m/z: 633.2 (M+1).

### Example 61d

Compound 73d was prepared following the procedure for compound 45a. m/z: 619.2 (M+1).

### Example 61e

Compound 73e was prepared following the procedure for compound 45a. m/z: 619.2 (M+1).

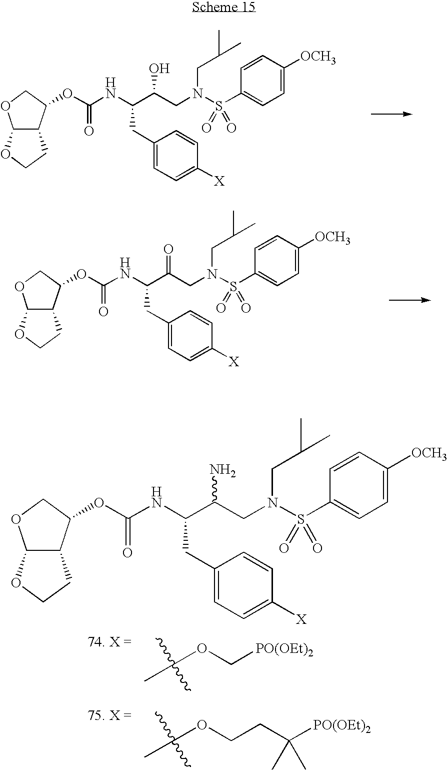

### Example 62

Compound 74 (70 mg) was synthesized following the procedure for compound 50a. m/z: 728.1 (M+1), 750.2 (M+Na).

### Example 63

Compound 75 (57 mg) was synthesized following the previous procedure. m/z: 784.2 (M+1), 806.3 (M+Na).

### Example 64

The following illustrate representative pharmaceutical dosage forms, containing a compound of the invention (‘Compound X’), for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |
| (ii) Tablet 2 | mg/tablet |
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 01 N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents (including U.S. patent application Ser. No. 11/030,632, which was published on 21 Jul. 2005 as United States Patent Application Publication Number US 2005/0158713) are incorporated by reference herein in their entirety, as though individually incorporated by reference. The invention has been described with reference to various specific and preferred embodiments and techniques. However, it should be understood that many variations and modifications may be made while remaining within the spirit and scope of the invention.

What is claimed is:

1. A compound of Formula (I): 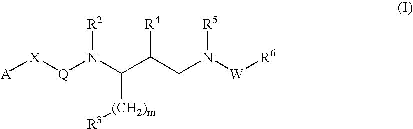
or a pharmaceutically acceptable salt thereof, wherein:
A is heteroaryl or a group having a formula: 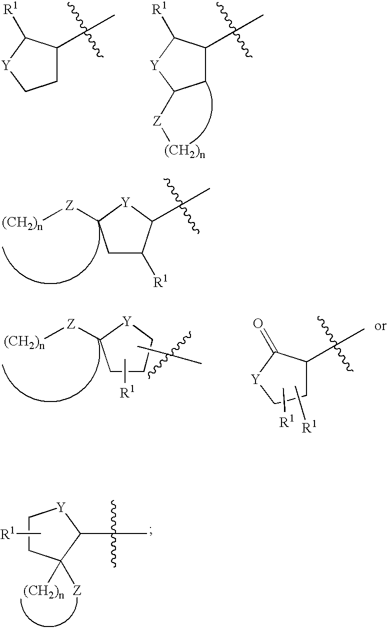
each R¹ is independently H, alkyl, alkenyl, alkynyl, cycloalkyl, cycloalkylalkyl, aryl, aralkyl, heterocycloalkyl, heterocycloalkylalkyl, heteroaryl, or heteroaralkyl, which R¹ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio;
Y and Z, the same or different, are independently selected from the group consisting of CH₂, O, S, SO, SO₂, NR⁸, R⁸C(O)N, R⁸C(S)N, R⁸OC(O)N, R⁸OC(S)N, R⁸SC(O)N, R⁸R⁹NC(O)N, and R⁸R⁹NC(S)N;
n is an integer from 1 to 5;
X is a covalent bond, CHR¹⁰, CHR¹⁰OCH₂, CH₂CHR¹⁰, O, NR¹⁰, or S;
Q is C(O), C(S), or SO₂;
R² is H, alkyl, alkenyl, or alkynyl;
m is an integer from 0 to 6;
R³ is cycloalkyl, heterocycloalkyl, aryl, or heteroaryl, which R³ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, (R¹⁰O)₂P(═O)—, (R¹⁰O)₂P(═O)alkyl, (R¹⁰O)₂P(═O)alkoxy, and heteroaralkylthio; wherein any cycloalkyl, heterocycloalkyl, aryl, and heteroaryl of the one or more substituents is optionally substituted with one or more halogen, cyano, nitro, carboxy, hydroxy, alkyl, haloalkyl, haloalkoxy, amino, alkylamino, alkanoylamino, alkoxy, alkylthio, or alkylamino;
R⁴ is ═NRₐ, or NRₐR_{b}; wherein Rₐ and R_{b} are each independently —V—R_{c}; wherein each V is independently a direct bond or —C(═O); and each R_{c} is independently H, hydroxy, alkyl, alkoxy, alkenyl, or alkynyl, and is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio;
R⁵ is H, C₁-C₆ alkyl, C₂-C₆ alkenyl, or (CH₂)_{q}R⁴, wherein q is an integer from 0 to 5; and R⁶ is cycloalkyl, heterocycloalkyl, aryl, or heteroaryl which R⁶ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio; or R⁵ and R⁶, together with the N—W bond of formula (I) comprise a macrocyclic ring which optionally comprises one or more additional heteroatoms in the macrocyclic ring;
R⁸ and R⁹ are each H, alkyl, alkenyl, or alkynyl;
each R¹⁰ is independently H, alkyl, alkenyl, or alkynyl;
R¹⁴ is cycloalkyl, heterocycloalkyl, aryl, or heteroaryl, which R¹⁴ is optionally substituted with one or more substituents independently selected from the group consisting of halogen, cyano, nitro, carboxy, hydroxy, alkyl, haloalkyl, haloalkoxy, amino, alkylamino, alkanoylamino, alkoxycarbonylaminoalkyl, alkoxy, alkylthio, alkylamino, cycloalkyl, cycloalkylalkyl, heterocycloalkyl, heterocycloalkylalkyl, aryl, aryloxy, arylcarbonyl, arylcarbonyloxy, arylamino, arylthio, aralkyl, aryloxyalkyl, arylaminoalkyl, aralkoxy, (aryloxy)alkoxy, (arylamino)alkoxy, (alkanoylamino)alkoxy, (arylthio)alkoxy, aralkylamino, (aryloxy)alkylamino, (arylamino)alkylamino, (arylthio)alkylamino, aralkylthio, (aryloxy)alkylthio, (arylamino)alkylthio, (arylthio)alkylthio, heteroaryl, heteroaryloxy, heteroarylamino, heteroarylthio, heteroaralkyl, heteroaralkoxy, heteroaralkylamino, and heteroaralkylthio; and
W is C(O), C(S), S(O), or SO₂.

2. The compound of claim 1 wherein A has the formula:

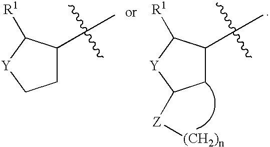

3. The compound of claim 1 wherein: Q is C(O); R² is H; and W is SO₂.

4. The compound of claim 1 wherein the compound of Formula (I) has the following formula:

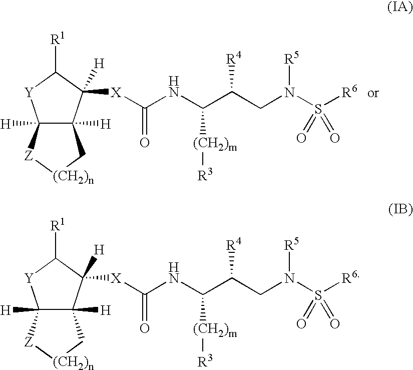

5. The compound of claim 1 wherein the compound of Formula (I) has the following formula: 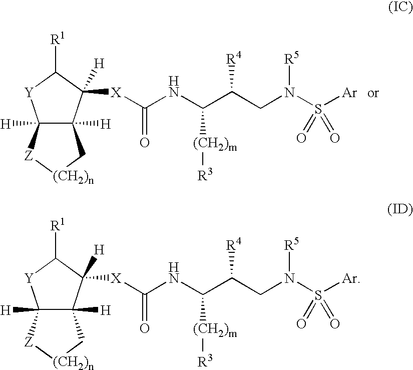
wherein Ar is a phenyl which is optionally substituted with a substituent selected from the group consisting of methyl, amino, hydroxy, methoxy, methylthio, hydroxymethyl, aminomethyl, and methoxymethyl.

6. The compound of claim 1 wherein R³ is thiophene or phenyl that is optionally substituted.

7. The compound of claim 1 wherein R³ is thiophene, phenyl, 4-[2-(tert-butoxycarbonylamino)ethoxy]phenyl, or 4-(2-methyl-1,3-thiazol-4-ylmethyloxy)phenyl.

8. The compound of claim 1 wherein R⁵ isisopropyl, isobutyl, butyl, benzyl, or phenethyl, cyclopropylmethyl, cyclopentylmethyl, or 2-ethylbytyl.

9. The compound of claim 1 wherein R⁶ is phenyl that is optionally substituted with methoxy, isobutyl, amino, fluoro, trifluoromethyl, trifluoromethoxy, acylamino, benzyloxy, ethoxy, isopropoxy, or 2-fluoroethoxy.

10. The compound of claim 1 wherein R⁶ is phenyl that is substituted at the 4-position with methoxy, isobutyl, amino, fluoro, trifluoromethyl, trifluoromethoxy, acylamino, benzyloxy, ethoxy, isopropoxy, or 2-fluoroethoxy.

11. The compound of claim 1 wherein the compound of Formula (I) has the following formula: 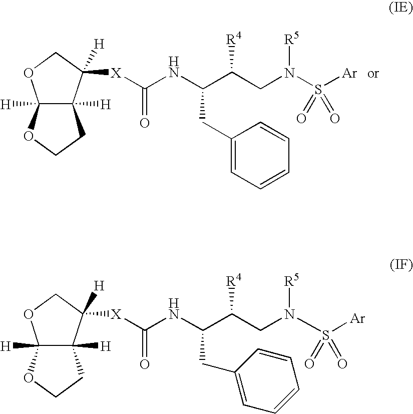
wherein Ar is a phenyl which is optionally substituted with a substituent selected from the group consisting of methyl, amino, hydroxy, methoxy, methylthio, hydroxymethyl, aminomethyl, and methoxymethyl.

12. The compound of claim 4 wherein X is oxygen.

13. The compound of claim 4 wherein R₅ is isobutyl.

14. The compound of claim 5 wherein Ar is phenyl substituted at the para position.

15. The compound of claim 5 wherein Ar is phenyl substituted at the meta position.

16. The compound of claim 5 wherein Ar is phenyl substituted at the ortho position.

17. The compound of claim 5 wherein Ar is selected from the group consisting of 4-aminophenyl, 4-methylphenyl, 4-methoxyphenyl, 3-methoxyphenyl, and 3-hydroxymethylphenyl.

18. The compound of claim 1 wherein the compound of Formula (I) has the following formula:

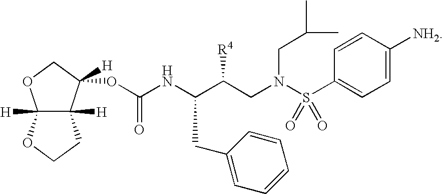

19. The compound of claim 1 wherein A has the formula:

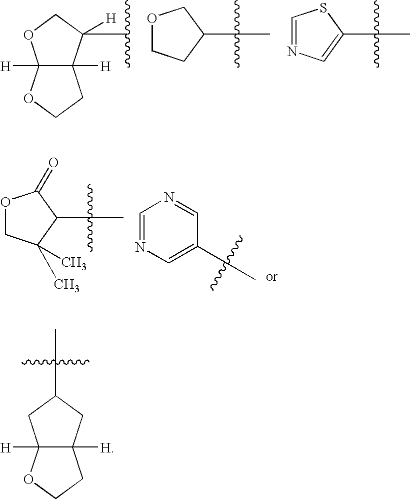

20. The compound of claim 1 wherein X is O.

21. The compound of claim 1 wherein Q is C(O); R² is H; and W is SO₂.

22. The compound of claim 1 wherein R³ is phenyl, 4-benzyloxyphenyl, 4-hydroxyphenyl, 4-methoxyphenyl, 4-(2-methylthiazol-4-ylmethoxy)phenyl, 4-[2-(tert-butoxycarbonylamino)ethoxy]phenyl, 4-[2-(methoxycarbonylamino)ethoxy]phenyl, 4-[2-methyl-2-(dimethylphosphono)propoxy]phenyl, 5-cyano-2-thienyl, or 4-(diethylphosphonomethoxy)phenyl.

23. The compound of claim 1 wherein R⁴ is amino, hydroxyimino, hydroxyamino, or methylamino.

24. The compound of claim 1 wherein R⁵ is 2-methylpropyl, butyl, propyl, phenyl, phenethyl, cyclopentylmethyl, or 2-ethylbutyl.

25. The compound of claim 1 wherein R⁶ has the formula:

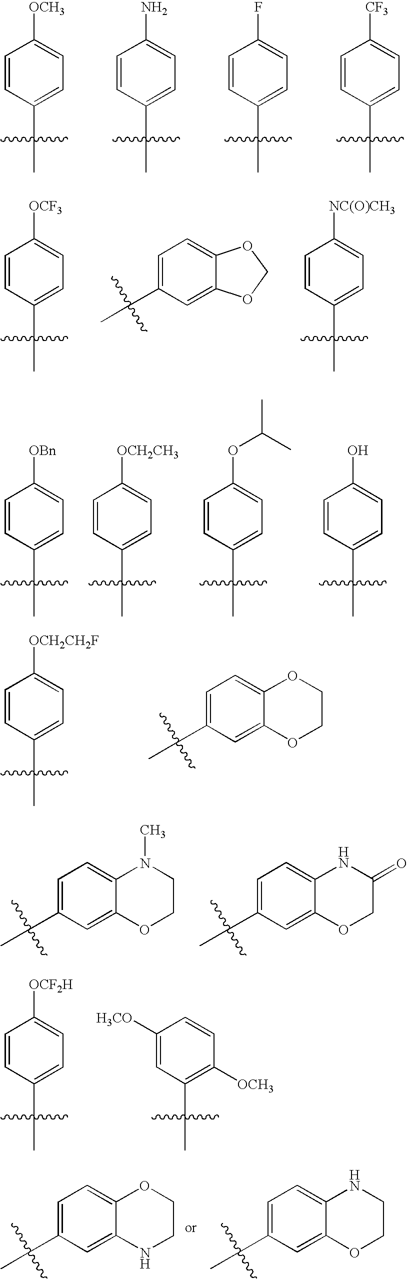

26. A pharmaceutical composition comprising a compound as described in claim 1 and a pharmaceutically acceptable carrier.

27. A method for inhibiting the activity of a protease comprising contacting the protease with an effective inhibitory amount of a compound as described in claim 1.

28. A method for reducing the likelihood that a disease-causing entity will develop drug resistance comprising, administering an effective amount of a compound as described in claim 1 to an animal that is infected with the disease causing entity.

29. The method of claim 28 wherein the disease-causing entity is a virus.

30. The method of claim 29 wherein the virus is HIV.

31. The use of a compound as described in claim 1 to prepare a medicament useful for inhibiting the activity of a protease in an animal.

32. The use of a compound as described in claim 1 to prepare a medicament useful for reducing the likelihood that a disease-causing entity will develop drug resistance in an animal.

33. The use of a claim 32 wherein the disease-causing entity is a virus.

34. The use of claim 33 wherein the virus is HIV.
